(19) United States
(12) Patent Application Publication
(10) Pub. No.: US 2016/0101188 A1
(43) Pub. Date: Apr. 14, 2016

(54) NOVEL NANOCARRIER DELIVERED CANCER CHEMOTHERAPEUTIC AGENTS

(71) Applicant: UNIVERSITY OF CINCINNATI, Cincinnati, OH (US)

(72) Inventors: Vladislav Alexander Litosh, Cincinnati, OH (US); Kayla M. Borland, Cincinnati, OH (US); Matthew P. Burke, Cincinnati, OH (US); Julia N. Tolstolutskaya, West Chester, OH (US)

(21) Appl. No.: 14/894,826

(22) PCT Filed: May 30, 2014
(86) PCT No.: PCT/US2014/040332

§ 371 Date: Nov. 30, 2015

Related U.S. Application Data
(60) Provisional application No. 61/829,675, filed on May 31, 2013.

(51) Int. Cl. A61K 47/48; A61K 31/7072; C07H 19/02; A61K 39/395

(52) U.S. Cl. CPC A61K 47/48092; A61K 47/48969; A61K 39/39558; A61K 47/484; A61K 31/7072; C07H 19/02; A61K 47/48884

(57) ABSTRACT

Compositions and methods for treating cancer in a subject are described herein. The composition includes modified nucleobases and nucleosides that are converted in the cell to nucleotides that are incorporated into growing DNA and result in termination of DNA elongation. The nucleobases and nucleotides are incorporated with a drug delivery system (DDS). The DDS includes β-cyclodextrin. The nucleobases and nucleotides are conjugated to the β-cyclodextrin by an acid labile linker that releases the nucleobases and nucleotides in the acidic environment of cancer cells. The DDS may also include a targeting ligand that targets the DDS/nucleobase or nucleotide conjugate to cancer cells. The DDS/nucleobase or nucleotide conjugate may self form into nanoparticles and may be administered to a subject with cancer in an amount effective to treat said cancer.

### RELATED APPLICATION

The Present application claims priority to U.S. Ser. No. 61/829,675 filed May 31, 2013, the disclosure of which is hereby incorporated herein by reference in its entirety.

### FIELD OF THE INVENTION

The present invention relates to therapeutic agents and methods and in particular, therapeutic agents and method useful in the treatment of cancer.

### BACKGROUND OF THE INVENTION

Cancer is one of the most sinister diseases known to mankind, claiming more than half-a-million lives in the U.S. alone in 2009. Chemotherapy remains a major path for treating most types of cancer, especially if the tumor is inoperable. However, the delivery of an effective doze of a drug to the tumor site while keeping the harmful side effects to the minimum remains the main challenge of cancer chemotherapy.

Contrary to normal cells, cancer cells undergo rapid, abnormal, and uncontrolled division, which warrants a constant need for DNA production. Interfering with this process therefore affects them preferentially, and represents a plausible approach to cancer chemotherapy. Among the existing chemotherapeutic drugs that target DNA of neoplastic cells are alkylators (e.g. nitrogen mustards, ethyleneimines, duocarmicines), intercalators (e.g. dactinomycin, quinoxalines) and DNA strand breakers (e.g. bleomycin, daunomycin, enediynes). The therapeutic doses of these drugs, however, are sufficiently high to cause severe side effects, sometimes lethal, connected to the damage inflicted upon healthy tissues. Although certain nucleotide analogs are used in treatment of cancer, their effect is based on inactivation of enzymes involved in DNA synthesis (e.g. 5-fluoro-2′-deoxyuridine) or interfering with nucleotide synthesis (e.g. 6-thioguanine), and the required dosage is also relatively high.

DNA sequencing by synthesis (SBS) utilizes modified nucleotides that are incorporated into the growing DNA strand by polymerases during the polymerase chain reactions (PCR) to result in termination of the growth of the strand into which the modified nucleotide is incorporated. The therapeutic potential of nucleotide species SBS have not previously been unexplored, mainly due to their poor incorporation by natural polymerases compared to natural nucleotides, which would require unacceptably high dosage. Recently, terminators for SBS were discovered that are incorporated into growing DNA strand by natural polymerases more efficiently than the corresponding natural nucleotides, at the same time terminating further DNA synthesis by obstructing the subsequent nucleotide incorporation. However, the chemotherapeutic potential of these terminators has not previously been explored.

Another challenge with developing new chemotherapeutic agents is finding an appropriate drug delivery system (DDS) to carry the chemotherapeutic agents specifically to the tumor. An ideal DDS needs to prevent the active drug from premature release during blood circulation, yet to provide reasonably expedient release at tumor site. Nanotechnology has the promise to satisfy these requirements by using nanoparticles for delivery of drugs, including chemotherapeutic agents. Micelles and liposomes have received a lot of attention as anti-cancer drug nanocarriers for their ability to encapsulate various chemotherapeutics, often sparingly soluble in water, but these systems have major drawbacks due to their instability in vivo and the lack of tunable triggers for drug release. Cyclodextrin-based carrier systems, on the other hand, do not suffer such disadvantages. An example of β-cyclodextrin polymer-camptothecin conjugate nanoparticle has recently been reported, although the drug is released slowly, and passive targeting is effected.

### SUMMARY OF THE INVENTION

Modified nucleobases and nucleosides undergo cellular uptake and within cells are readily converted to nucleotides, both in vivo and in vitro. Modified nucleotides may be incorporated into the growing DNA strand and if they include a bulky group, such as a branched alkyl (e.g. isopropyl or tert-butyl) attached to the α-benzylic carbon, result in termination of elongation of the DNA strand.

An aspect of the present invention includes modified nucleosides, as well as modified nucleobases, that function as prodrugs exhibiting at least cytostatic and preferably, cytotoxic activity in cancer cells. The novel prodrugs that are expected to convert into actual chemotherapeutic agents inside the cancer cell through the series of enzymatic transformations that convert the nucleobase or nucleoside into nucleotides that are incorporated into growing DNA strands by polymerases within the cell and result in termination of DNA elongation. Due to outstanding incorporation properties of their 5′-triphosphates, these agents have high potency, and as a result be effective in lower therapeutic doses than traditional chemotherapeutic drugs that are currently on the market.

Another aspect of the invention is a drug delivery system (DDS) to carry the modified nucleobases and nucleosides specifically to the tumor. The DDS prevents the modified nucleobases and nucleosides from premature release during blood circulation, yet provides reasonably expedient release at tumor site as well as providing adequate solubility, high drug loading capacity, and a long residence time before renal clearance. Cyclodextrin-based (CD) carrier systems have robust, well-defined chemical structure, with many potential sites for covalent attachment. The invaluable properties of these cyclic oligosaccharides include low toxicity, low immunogenicity, and hydrophobicity of the CD cavity making it possible to form a non-covalent host-guest inclusion complex with the entire drug or a portion of it (presumably, hydrophobic), which provides additional protection of the drug from biodegradation. Furthermore, organization of CDs into supramolecular entities, such as linear co-polymers (CDP) capable of self-assembling into multi-strand nanoparticles, which are sufficiently large to avoid renal clearance, but disassemble upon drug release followed by excretion.

The prodrug nucleobases and nucleosides may be conjugated to the CD by an acid labile linker that results in the release of the prodrug in the acidic environment of cancer cells. The DDS may also be functionalized to include a targeting ligand that targets molecules specifically or preferentially expressed by cancer cells.

### BRIEF DESCRIPTION OF THE DRAWINGS

The accompanying drawings, which are incorporated in and constitute a part of this specification, illustrate embodiments of the invention and, together with a general description of the invention given above and the detailed description of the embodiments given below, serve to explain the embodiments of the invention.

FIG. 1 is a schematic representation of the proposed mechanism of action of a modified nucleoside in accordance with aspects of the invention.

FIG. 2 is a concentration response curve for a modified nucleoside in accordance with aspects of the invention.

FIG. 3 is a graph showing an increase in a marker indicating double stranded DNA breakage in cell exposed to a modified nucleoside in accordance with aspects of the invention.

### DETAILED DESCRIPTION OF THE INVENTION

An aspect of the invention is directed to modified nucleobases and nucleosides that may be incorporated into a drug delivery system and function as chemotherapeutic agents by disrupting DNA synthesis. The modified nucleobases and nucleosides are analogs of naturally occurring nucleobases cytosine (C), guanine (G), adenine (A), and thymine (T). A nucleobase coupled to a sugar, such ribose or 2′-deoxydeoxyribose, is referred to as a nucleoside. A nucleoside bearing one or more phosphate groups attached at the 5′ or 3′ hydroxy group is referred to as a nucleotide. The modified nucleobases or base-modified nucleosides are prodrugs that are presumably metabolized into nucleotides that serve as chemotherapeutic drugs due to their ability to incorporate into a DNA replication fork of the DNA of cancer cell, thereby resulting in disruption of DNA synthesis, which eventually leads to cell death.

The structural formulas for the modified nucleobases and nucleosides in accordance with embodiment are illustrated below. Formula 1a,b refers thymidine analogs, with 1a referring to the nucleobase and 1 b referring to the nucleoside. Formula 2a,b refers cytosine analogs, with 2a referring to the nucleobase and 2b referring to the nucleoside. Formula 3a,b refers to a first adenine analogs, with 3a referring to the nucleobase and 3b referring to the nucleoside. Formula 4a,b refers to 7-deaza-adenine analogs, with 4a referring to the nucleobase and 4b referring to the nucleoside. Formula 5a,b refers to a 7-deaza-guanine analogs, with 5a referring to the nucleobase and 5b referring to the nucleoside.

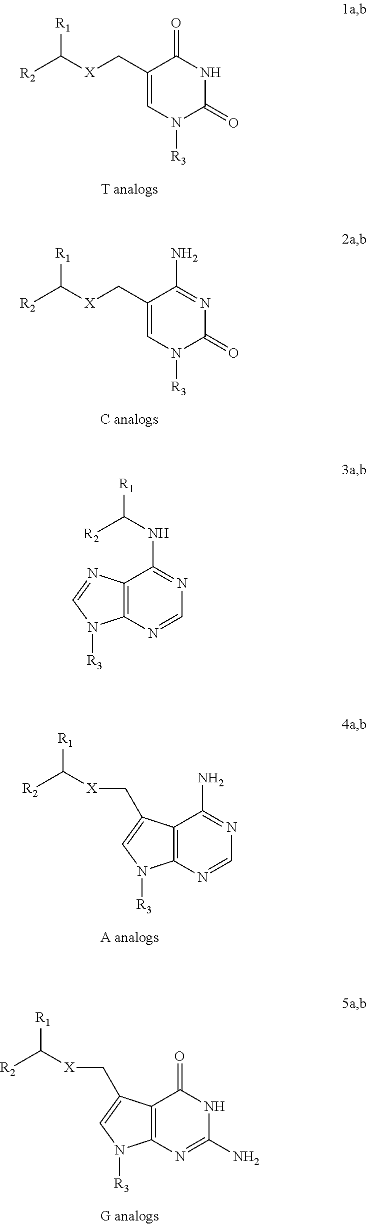

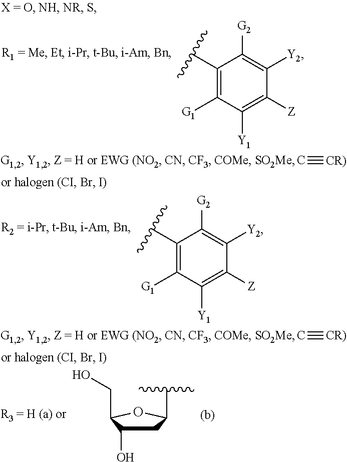

In an aspect of the invention, the modified nucleosides are incorporated into a drug delivery system (DDS). In an embodiment, the DDS includes the modified nucleotide or nucleobase conjugated to a cyclodextrin (such as β-cyclodextrin) by an acid labile linker. The acid labile linker releases the modified nucleobase or nucleoside when exposed to an acidic pH, such as the acidic environment found in and around many tumor cells. Exemplary acid labile linkers include an acetyl linker such as formed by a benzaldehyde diacetal derivative. Hydrazone is another exemplary acid labile linker that may be useful in accordance with embodiments of the invention.

The DDS may also optionally functionalized to include a targeting ligand that will allow the DDS to target tissues expressing receptors for the ligand. For example, cancerous cells express or over express certain cancer specific receptors capable of interacting with proteins, carbohydrates, and lipids, and antibodies, that are either not expressed or are not expressed to the same levels in normal tissues. Targeting ligands include compounds that specifically bind to cancer specific receptors. Targeting ligands may include small molecules, peptides, proteins, lipids, carbohydrates, and combinations thereof. Exemplary targeting ligands include folate, transferrin, RGD peptide, anisimide, and numerous cancer specific antibodies, such a monoclonal antibodies like trastuzumab. The targeting ligands may be conjugated to the DDS, such as by covalent bonding to one of the hydroxyl groups on the cyclodextrin.

The targeting ligand may also promote entry of the DDS conjugate into the cell via endocytosis. The acidity of the endocytotic vesicle will promote the release of the modified nucleobase or nucleoside from the DDS conjugate via the cleavage of the acid labile linker. Once released into the interior of the cell, the modified nucleosides and nucleobases are enzymatically converted to nucleotides, which may then be incorporated by polymerases into the replicating DNA strands. The modifications on the nucleotides terminate DNA strand elongation, which has a cytostatic or cytotoxic effect on rapidly dividing cells, such as cancer cells.

Another aspect of the invention is directed to nanoparticles formed from the modified nucleobase/nucleoside/DDS conjugates. As discussed in greater detail below, cyclodextrin based co-polymers undergo self-assembly in aqueous solutions to form nanoparticles. The nanoparticles are of a size sufficient to prevent the nanoparticles from being rapidly removed from the blood in the kidneys and to prevent the nanoparticles from penetrating normal capillaries. To this end, the nanoparticles have a diameter of at least about 8 nm and preferably of at least about 10 nm. Capillaries in cancerous tissues may be penetrated by particles having a diameter of up to around 100 nm. Thus, the nanoparticles formed in accordance with the present invention have a diameter ranging from about 8 nm to about 100 nm or from about 10 nm to about 100 nm, which will prevent them from penetrating the wall of a normal capillary, but they will penetrate the wall of a highly porous tumor capillary.

The DDS/nucleobase or nucleoside conjugate should increase the efficiency of the drug and minimize the harmful side effects to patients due to the non-specific cellular uptake (i.e. when the chemotherapeutic agents penetrate the normal, healthy cells). Thus, another aspect of the invention is directed to the treatment of subject with cancer using a modified nucleobase or nucleoside and in particular, by administering to a subject having cancer a modified nucleobase or nucleoside conjugated to a DDS or by administering to the subject a nanoparticle formed from the conjugate in an amount effective to cause cytotoxicity in a tumor cell in the subject. Those of ordinary skill will be able to determine the appropriate route of administration, dose, and dosing regimen without undue experimentation as those types of studies are routine.

Synthetic Procedures

All chemicals, reagents, and solvents were purchased from Sigma-Aldrich Inc., TCI, and Fisher Scientific, Inc, and used as received unless stated otherwise. All reactions were carried out under an atmosphere of dry argon in oven-dried glassware. Indicated reaction temperatures refer to those of the reaction bath, while room temperature (rt) is noted as 25° C. Pure reaction products were typically dried under high vacuum in the presence of phosphorus pentoxide. Analytical thin layer chromatography (TLC) was performed with glass backed silica plates (5×20 cm, 60 Å, 250 μm). Visualization was accomplished using a 254 nm UV lamp. ¹H and ¹³C NMR spectra were recorded on either a Bruker Avance 400 MHz spectrometer or Bruker DPX 500 MHz spectrophotometer. Chemical shifts are reported in ppm with tetramethylsilane as standard. Data are reported as follows: chemical shift, number of protons, multiplicity (s=singlet, d=doublet, dd=doublet of doublet, t=triplet, q=quartet, b=broad, m=multiplet, abq=ab quartet), and coupling constants. High resolution mass spectral data were collected on a Shimadzu Q-TOF 6500.

Synthesis of Alcohols.

Benzyl alcohol, 1-phenylethanol, 1-phenyl-2-methyl-1-propanol, 1-phenyl-2,2-dimethyl-1-propanol, 2,4-dimethyl-3-pentanol, 2-nitrobenzyl alcohol, and 1-(2-nitrophenyl)ethanol were available commercially. 1-(2-Nitrophenyl)-2-methyl-1-propanol,¹⁴ 1-(2-nitrophenyl)-2,2-dimethyl-1-propanol,¹⁴ diphenylmethanol,²³ 2,2,4,4-tertramethyl-3-pentanol (di-tert-butylcarbinol),²⁴ 1-phenyl-1-heptanol,²⁵ 1-(3-methoxy)phenyl-2,2-dimethyl-1-propanol,²⁶ 1-(4-methoxy)phenyl-2,2-dimethyl-1-propanol,²⁶ 1-(2-bromophenyl)-2,2-dimethyl-1-propanol,²⁷ 2-nitro-(α-phenyl)benzyl alhocol,²⁸1-(2,6-dinitrophenyl)-2,2-dimethyl-1-propanol¹⁵1-(2-nitrophenyl)-(phenyl)methanol,¹⁵ and 1-(4-iodo-2-nitrophenyl)-2,2-dimethyl-1-propanol¹⁵ were prepared in accordance with literature protocols. 1-Phenyl-3,3-dimethylbutanol²⁹ and α-cyclohexyl-benzyl alcohol³⁰ were prepared by Grignard addition of phenylmagnesium bromide to 3,3-dimethylbutanal and cyclohexanecarboxaldehyde, respectively, whereas 1-(2-methoxyphenyl)-2,2-dimethyl-1-propanol³¹ and 1-(2-methylphenyl)-2,2-dimethyl-1-propanol³² were prepared by addition of tert-butylmagnesium chloride to o-anyzaldehyde and o-methylbenzaldehyde, respectively, and were identified according to their ¹H NMR data given in literature.

Preparation of Novel α-Substituted Benzyl Alcohols

Transmetallation of 1-iodo-2-nitrobenzene followed by reaction with aldehydes

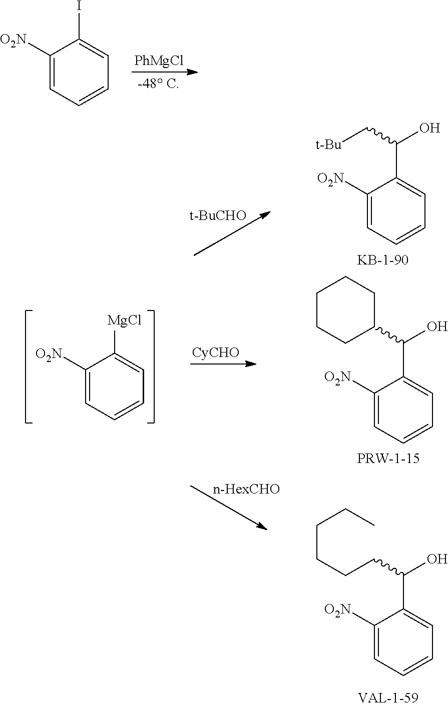

General Procedure for Transmetallation:

To a solution of 1-iodo-2-nitrobenzene in anhydrous tetrahydrofuran (0.4 M) cooled at minus 48° C. (dry ice-acetone bath) under argon atmosphere, phenylmagnesium chloride (2 M in THF, 1.0-1.1 eq.) was added at a rate to keep the temperature at or below minus 35° C. Upon completion of the addition the mixture was stirred for five minutes, then the appropriate aldehyde (1.1-1.5 eq.) was added. The mixture was allowed to gradually warm up to room temperature, then quenched with saturated aqueous NH₄Cl (ca 5 mL) and poured into ethyl acetate (25 mL). The organic layer was separated; aqueous layer was extracted three times with ethyl acetate (25 mL each). Combined organic extract was washed with water (20 mL), dried over anhydrous Na₂SO₄, concentrated under reduced pressure, and purified by silica gel column chromatography.

(±)-1-(2-nitrophenyl)-3,3-dimethyl-1-butanol (KB-1-90):

3.512 g (14.11 mmol) of 1-iodo-2-nitrobenzene and 1.7 mL (13.54 mmol) of cyclohexaldehyde were used. Column chromatography (hexane/ethyl acetate 20:1 to 10:1) afforded (RS)-1-(2-nitrophenyl)-3,3-dimethyl-1-butanol, 1.35 g, 45%, as light yellow oil. ¹H NMR (400 MHz, CDCl₃): δ 7.85 (dd, 1H, J=8.2, 1.4 Hz), 7.82 (dd, 1H, J=8.2, 1.4 Hz), 7.63 (dt, 1H, J=7.6, 1.4 Hz), 7.40 (m, 1H), 5.41 (m, 1H), 2.40 (d, 1H, J=4.2 Hz), 1.70 (AB dd, 1H, J=14.5, 8.9 Hz), 1.63 (AB dd, 1H, J=14.5, 2.6 Hz), 1.05 (s, 9H). ¹³C NMR (100 MHz, CD₃OD): δ 147.34 (C), 141.48 (C), 133.45 (CH), 128.37 (CH), 127.86 (CH), 124.16 (CH), 67.52 (CH), 51.55 (CH₂), 30.87 (C), 30.21 (CH₃). HRMS (TOF ES⁺) for [MNa]⁺C₁₂H₁₇NO₃Na calculated: 246.1106, observed: 246.1100.

(±)-(2-nitrophenyl)-cyclohexylmethanol (PRW-1-15):

1.16 g (4.678 mmol) of 1-iodo-2-nitrobenzene and 0.623 mL (5.143 mmol) of cyclohexaldehyde were used. Column chromatography (hexane/ethyl acetate 20:1 to 8:2) afforded (RS)-(2-nitrophenyl)-cyclohexylmethanol, 0.838 g, 76%, as light yellow oil. ¹H NMR (400 MHz, CDCl₃): δ 7.87 (dd, 1H, J=8.2, 1.3 Hz), 7.74 (dd, 1H, J=7.9, 1.5 Hz), 7.63 (dt, 1H, J=7.6, 1.3 Hz), 7.43 (dt, 1H, J=7.6, 1.5 Hz), 5.06 (d, 1H, J=6.3 Hz), 2.50 (s, 1H), 1.86 (m, 1H), 1.75 (m, 4H), 1.43 (d, 1H, J=12.7 Hz), 1.18 (m 4H), 0.90 (m, 1H). ¹³C NMR (100 MHz, CD₃OD): δ 148.62 (C), 138.60 (C), 132.92 (CH), 129.05 (CH), 127.98 (CH), 124.24 (CH), 73.45 (CH), 44.06 (CH), 29.79 (CH₂), 27.75 (OH₂), 26.30 (CH₂), 26.20 (CH₂), 25.97 (CH₂). HRMS (TOF ES+) for [MNa]⁺C₁₃H₁₇NO₃Na⁺ calculated: 258.1101, observed: 258.1102.

(±)-1-(2-nitrophenyl)-1-heptanol (VAL-1-59):

1.00 g (4.02 mmol) of 1-iodo-2-nitrobenzene and 0.636 mL (6.03 mmol) of heptaldehyde were used. Column chromatography (hexane/ethyl acetate 20:1 to 10:2) yielded (RS)-1-(2-nitrophenyl)-1-heptanol, 0.66 g, 63%, as light yellow oil. ¹H NMR (400 MHz, CDCl₃): δ 7.91 (d, 1H, J=8.2 Hz), 7.82 (d, 1H, J=7.9 Hz), 7.65 (t, 1H, J=7.7, Hz), 7.44 (t, 1H, J=7.7 Hz), 5.24 (m, 1H), 2.39 (s, 1H, J=7.7 Hz), 1.80 (m, 2H), 1.51 (m, 1H), 1.32 (m, 7H), 0.90 (m, 3H). ¹³C NMR (100 MHz, CD₃OD): δ 147.92 (C), 140.28 (C), 133.44 (CH), 128.04 (CH), 128.00 (CH), 124.31 (CH), 69.42 (CH), 38.19 (CH₂), 31.57 (CH₂), 29.01 (CH₂), 26.13 (CH₂), 22.60 (CH₂), 14.10 (CH₃).

Addition of Tert-Butylmagnesium Chlorides to Substituted Benzaldehydes

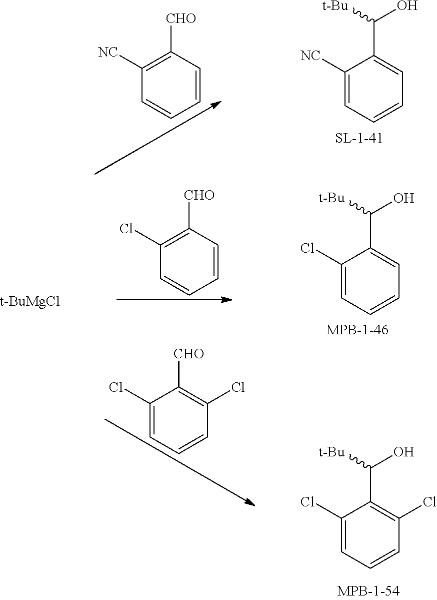

General Procedure for Grignard Addition:

To a solution of the appropriate aldehyde in anhydrous tetrahydrofuran (0.4 M) cooled at 0° C. (ice-water bath) tert-butylmagnesium chloride (2.0 M in THF, 1.0-1.2 eq) was added to the mixture at a rate to keep the temperature below 10° C. The mixture was allowed to gradually warm up to room temperature, then quenched with saturated aqueous NH₄Cl (ca 5 mL) and poured into ethyl acetate (25 mL). The organic layer was separated; aqueous layer was extracted three times with ethyl acetate (25 mL each). Combined organic extract was washed with water (20 mL), dried over anhydrous Na₂SO₄, concentrated under reduced pressure, and purified by silica gel column chromatography.

(±)-1-(2-cyanophenyl)-3,3-dimethyl-1-butanol (SL-1-41):

0.5 g (3.8 mmol) of 2-cyanobenzaldehyde and 1.9 mL (3.8 mmol) of tert-butylmagnesium chloride solution were used. Column chromatography (hexane/ethyl acetate 20:1 to 8:1) yielded (RS)-1-(2-cyanophenyl)-3,3-dimethyl-1-butanol, 0.097 g, 13%, as light yellow. ¹H NMR (400 MHz, CDCl₃): δ 7.77 (d, 1H, J=7.5), 7.37 (m, 3H), 5.03 (s, 1H), 0.90 (s, 9H). ¹³C NMR (100 MHz, CD₃OD): δ 145.18 (C), 131.28 (CH), 130.19 (C), 128.43 (CH), 123.61 (CH), 122.89 (CH), 90.31 (CH), 35.83 (C), 25.19 (CH₃).

(±)-1-(2-chlorophenyl)-3,3-dimethyl-1-butanol (MPB-1-46):

1.41 g (10 mmol) of o-chlorobenzaldehyde and 5 mL (12 mmol) of tert-butylmagnesium chloride solution were used. Column chromatography (hexane/ethyl acetate 20:1 to 10:2) yielded (RS)-1-(2-chlorophenyl)-3,3-dimethyl-1-butanol, 1.3 g, 92%, as light yellow. ¹H NMR (400 MHz, CDCl₃): δ 7.56 (AB dd, 1H, J=7.8, 1.8 Hz), 7.34 (AB dd, 1H, J=8.0, 1.4 Hz), 7.28 (dt, 1H, J=7.5, 1.3 Hz), 7.21 (AB dt, 1H, J=7.7, 1.8 Hz), 5.04 (s, 1H), 2.15 (br. s, 1H), 1.0 (s, 9H). ¹³C NMR (100 MHz, CD₃OD): δ 139.98 (C), 133.44 (C), 129.46 (CH), 129.10 (CH), 128.32 (CH), 126.28 (CH), 76.61 (CH), 36.91 (C), 25.82 (CH₃).

(±)-1-(2,6-dichlorophenyl)-3,3-dimethyl-1-butanol (MPB-1-54):

2 g (11.4 mmol) of 2,6-Dichlorobenzaldehyde and 6.86 mL (13.7 mmol) of tert-butylmagnesium chloride solution were used. Column chromatography (hexane/ethyl acetate 20:1 to 10:2) yielded (RS)-1-(2,6-chlorophenyl)-3,3-dimethyl-1-butanol as light yellow. ¹H NMR (400 MHz, CDCl₃, note: due to restricted rotation, H-3 and H-5 are inequivalent): δ 7.36 (dd, 1H, J=8.0, 1.4), 7.31 (dd, 1H, J=8.1, 1.4), 7.15 (t, 1H, J=8.0), 5.33 (d, 1H, J=11.2), 3.25 (d, 1H, J=11.2), 1.09 (s, 9H). ¹³C NMR (100 MHz, CDCl₃, note: due to restricted rotation, all aromatic carbons are inequivalent to one another): δ 136.48 (C), 136.01 (C), 133.79 (C), 130.63 (CH), 128.98 (CH), 128.54 (CH), 79.69 (CH), 39.17 (C), 27.35 (CH₃).

Synthesis of Nucleobases

Several examples of thymine nucleobase analogs (formula 1a) have been synthesized by treatment of 5-chlorouracil with appropriate alcohols at elevated temperatures (Scheme 1).

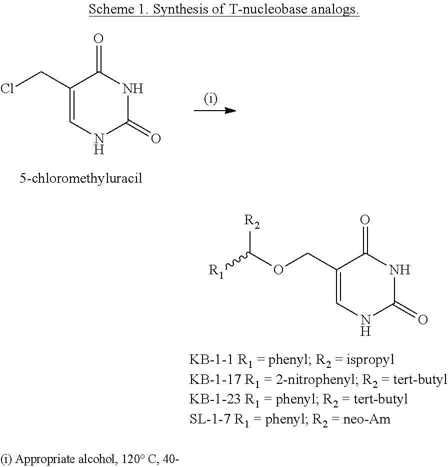

General Procedure.

5-Chloromethyluracil and appropriate alcohol (4-11 eq.) were heated neat at 120° C. for 0.5-3 hours under argon atmosphere. The mixture was cooled down to room temperature, dissolved in dichloromethane/methanol=20:1, and silica (ca 5 g) was added. The solvent was removed under reduced pressure and the solid was applied onto a silica gel column. Chromatography (SiO₂, CH₂Cl₂/MeOH=20:1) afforded the product as a powder.

### 5-[1-(2-nitrophenyl)-2,2-(dimethyl)propoxymethyl]uracil (KB-1-17)

Treatment of uracil (50 mg, 0.311 mmol) with 272 mg (1.3 mmol) of racemic α-tert-butyl-2-nitrobenzyl alcohol (2,2-dimethyl-1-(2-nitrophenyl)-1-propanol¹⁵) for 2.5 hours afforded after purification 32 mg of product (31%). ¹H NMR (400 MHz, DMSO-d₆) δ 11.09 (br. s, D₂O exchangeable) 10.86 (br. s, D₂O exchangeable), 7.88 (d, 1H, J=7.9 Hz), 7.70 (m, 2H), 7.56 (t, 1H, J=7.4 Hz), 7.41 (s, 1H), 4.79 (s, 1H), 4.08 (AB d, 2H, J=11.6 Hz), 3.94 (AB d, 2H, J=11.6 Hz), 0.79 (s, 9H). ¹³C NMR (100 MHz, CD₃OD) δ 160.04 (C), 151.73 (C), 150.85 (C), 141.28 (CH), 133.65 (C), 132.76 (CH), 130.08 (CH), 129.17 (CH), 124.22 (CH), 109.04 (C), 80.96 (CH), 64.42 (CH₂), 36.53 (C), 25.97 (CH₃). HRMS (ESI) for [MH]⁺ calculated: 334.13975, observed: 334.13977.

### 5-[1-phenyl-2-(methyl)propoxymethyl]uracil (KB 1-1)

Treatment of uracil (50 mg, 0.311 mmol) with 467 mg (3.114 mmol) of commercial racemic α-isopropylbenzyl alcohol (2-methyl-1-phenyl-1-propanol) for 0.5 hours afforded after purification 60 mg of product (70%). ¹H NMR (400 MHz, DMSO-d6) δ 11.10 (br. s, D₂O exchangeable) 10.83 (br. s, D₂O exchangeable), 7.28 (m, 6H), 4.03 (AB d, 1H, J=6.9 Hz), 3.95 (AB d, 2H, J=35.3, 12.0 Hz), 3.86 (AB d, 2H, J=35.3, 12.0 Hz), 1.84 (m, 1H), 0.89 (AB d, 3H, J=6.5) 0.68 (AB d, 3H, J=6.5 Hz). ¹³C NMR (100 MHz, CD₃OD) δ 164.08 (C), 151.71 (C), 141.50 (C), 140.16 (CH), 128.46 (CH), 127.70 (CH), 127.62 (CH), 109.93 (C), 86.55 (CH), 63.40 (CH₂), 34.60 (CH), 19.27 (CH₃), 19.03 (CH₃). HRMS (ESI) for [MH]⁺ calculated: 275.13902, observed: 275.13909.

### 5-[1-phenyl-2,2-(dimethyl)propoxymethyl]uracil (KB 1-23)

Treatment of uracil (57 mg, 0.355 mmol) with 660 mg (4.018 mmol) of commercial racemic α-tert-butylbenzyl alcohol (2,2-dimethyl-1-phenyl-1-propanol) for 1 hour afforded after purification 25 mg of product (24%). ¹H NMR (400 MHz, DMSO-d6) δ 11.09 (br. s, D₂O exchangeable) 10.80 (br. s, D₂O exchangeable), 7.31 (m, 6H), 4.05 (s, 1H), 3.97 (AB d, 2H, J=12.1 Hz), 3.82 (AB d, 2H, J=12.1 Hz), 0.82 (s, 9H). ¹³C NMR (100 MHz, CD₃OD) δ 164.05 (C), 151.71 (C), 140.06 (CH), 139.73 (C), 128.58 (CH), 127.93 (CH), 127.62 (CH), 109.90 (C), 88.61 (CH), 63.73 (CH₂), 35.60 (C), 26.52 (CH₃). HRMS (ESI) for [MH]⁺ calculated: 289.15467, observed: 289.15465.

### 5-[1-phenyl-3,3-(dimethyl)butoxymethyl]uracil (SL 1-7)

Treatment of uracil (50 mg, 0.311 mmol) with 121 mg (0.702 mmol) of α-neo-pentylbenzyl alcohol (3,3-dimethyl-1-phenyl-1-propanol, was obtained by Grignard addition of phenylmagnesium chloride to 3,3-dimethylbutanal) for 3 hours afforded after purification 6 mg of product (6%). ¹H NMR (400 MHz, CD₃OD) δ 7.31 (s, 6H), 4.53 (dd, 1H, J=8.8, 3.3 Hz), 4.00 (s, 2H), 1.81 (dd, 1H, J=14.5, 8.8 Hz), 1.44 (dd, 1H, J=14.5, 3.3 Hz), 0.97 (s, 9). HRMS (ESI) for [MNa]⁺ calculated: 325.15226, observed: 325.15232.

Additional thymine nucleobase analogs (Formula 1a) will be synthesized likewise: 5-chlorouracil will be treated with an appropriate amine or an ionized thiol (Scheme 2).

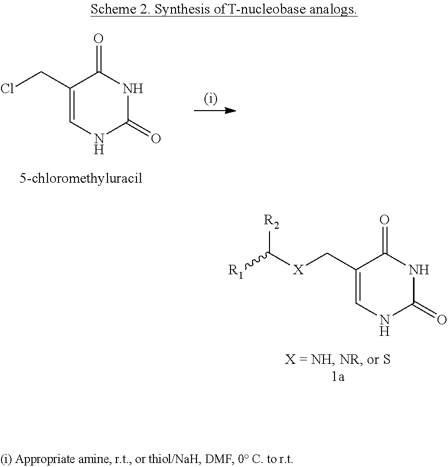

5-Substituted cytosine nucleobase analogs (Formula 2a) will be synthesized starting from the T-nucleobases. Briefly, N¹-acetylation¹⁹ followed by transformation of the uracil fragment into cytosine will furnish the desired C-nucleobases (Scheme 3).

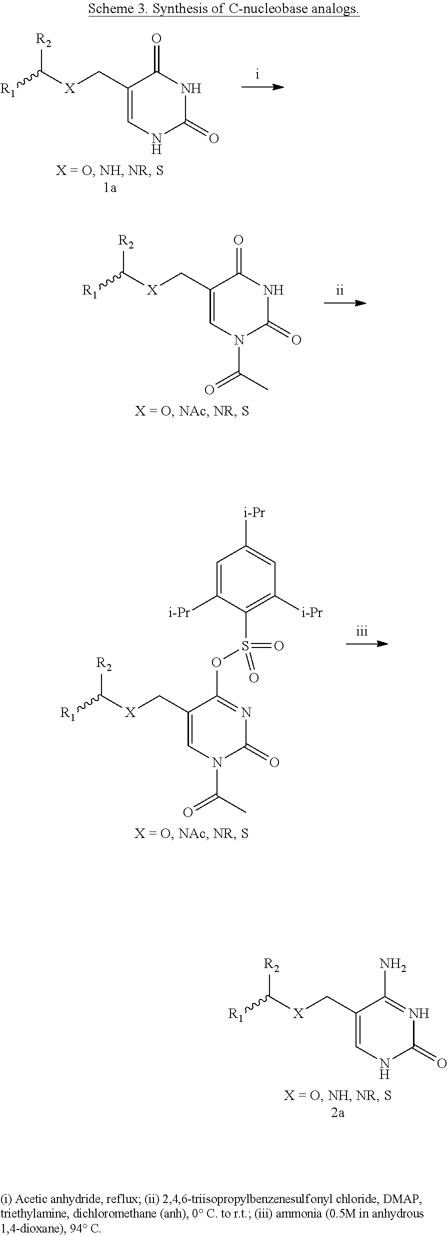

N-Alkylated adenine nucleobase analogs (Formula 3a) will be synthesized as follows: hypoxanthine will undergo N-acetylation¹⁹ followed by reaction with an appropriate amine mediated by (benzotriazol-1-yloxy)tris(dimethylamino)phosphonium hexafluorophosphate (BOP), as reported previously.¹⁴ Subsequent removal of the 9-acetyl group will furnish the desired N-alkyl nucleobases (Scheme 4).

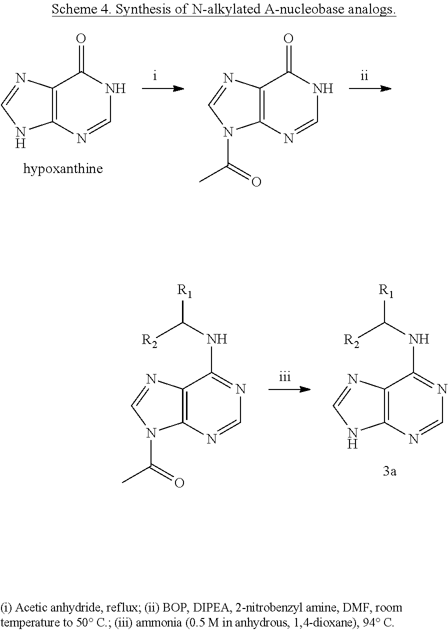

7-Deaza-7-substituted adenine nucleobase analogs (Formula 4a) will be synthesized starting from commercially available 6-chloro-7-iodo-7-deazapurine following an already established routine for nucleosides. Briefly, Pd-mediated CO insertion in methanol will provide 7-deaza-7-methyl carboxylate. Protection of the N-9⁹-position followed by selective ester reduction will yield 7-deaza-7-hydroxymethyl derivative. Subsequently, the 7-hydroxyl will be converted into 7-chloro derivative followed by reaction with a desired nucleophile. Treatment with ammonia at elevated temperature will effect the replacement of 6-chloride with the amino group, as well as the removal of N⁹-acyl protection (Scheme 5).

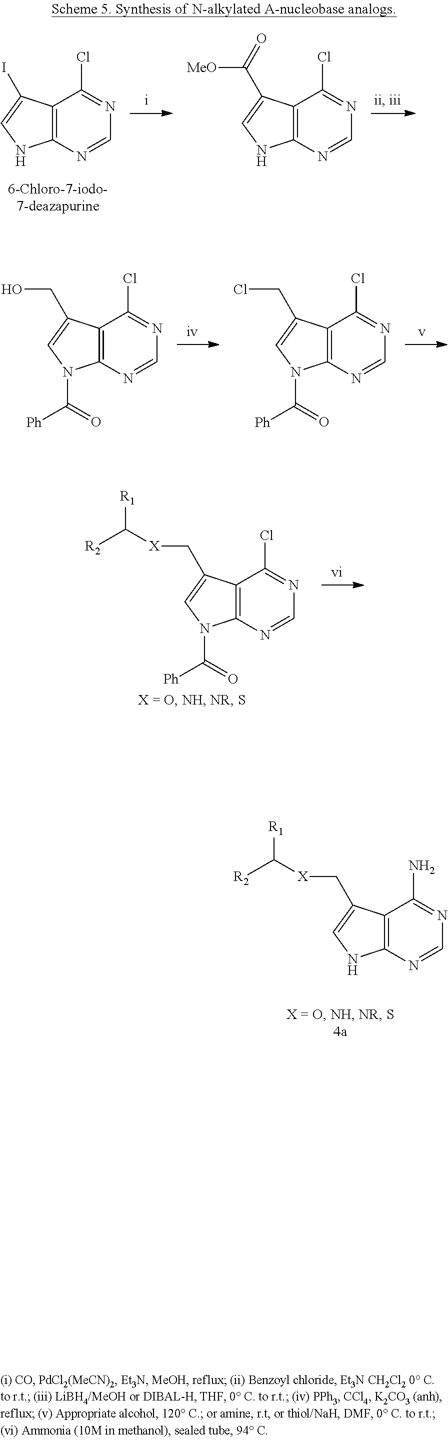

7-Deaza-7-substituted guanine nucleobase analogs (Formula 5a) will be synthesized starting from commercially available 4-chloro-7H-pyrrolo[2,3-d]pyrimidin-2-amine following an already established routine for nucleosides. Briefly, protection of the N⁹-position and 2-amino groups will be followed by treatment with N-iodosuccinimide to produce the 7-iodo derivative. Subsequently, Pd-mediated CO insertion in methanol will provide 7-deaza-7-methyl carboxylate, which will be followed by selective ester reduction will yield 7-deaza-7-hydroxymethyl derivative. Then the 7-hydroxyl group will be converted into 7-chloro followed by reaction with a desired nucleophile. Treatment with DABCO and then sodium acetate in DMF will replace the C⁶—Cl bond with C⁶═O. Removal of the N-protecting groups will furnish the desired nucleoside (Scheme 6).

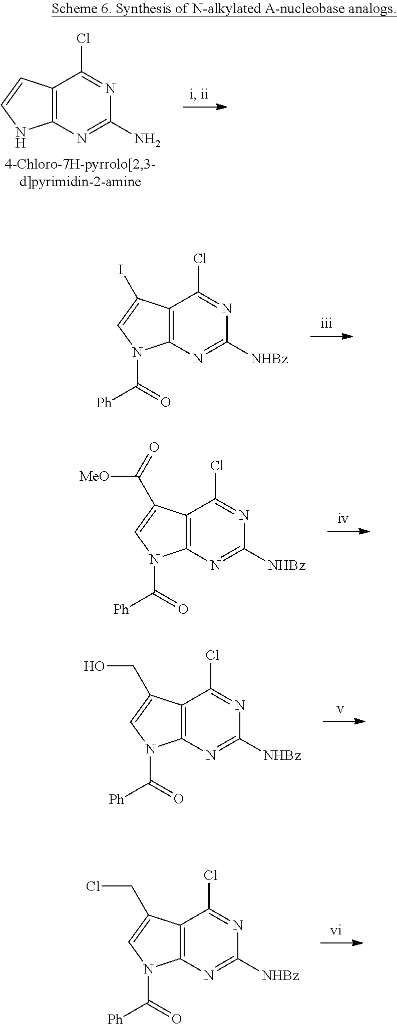

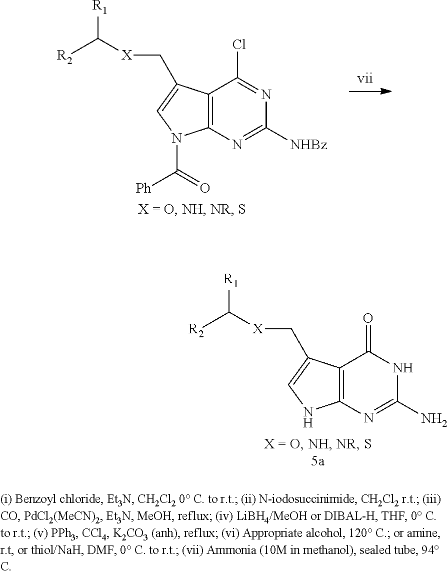

### Synthesis of Nucleosides

### Synthesis of T-Nucleoside Analogs

Several examples of thymidine nucleoside analogs (Formula 1b) have been synthesized similarly to the previously described procedure^{14,15} by treatment of N³-tert-butyloxycarbonyl-5-bromomethyl-3′,5′-bis-O-tert-butyldimethylsilyl-2′-deoxyuridine (5-BrCH₂-dU) with appropriate alcohols at elevated temperatures followed by the removal of TBS groups (Scheme 7).

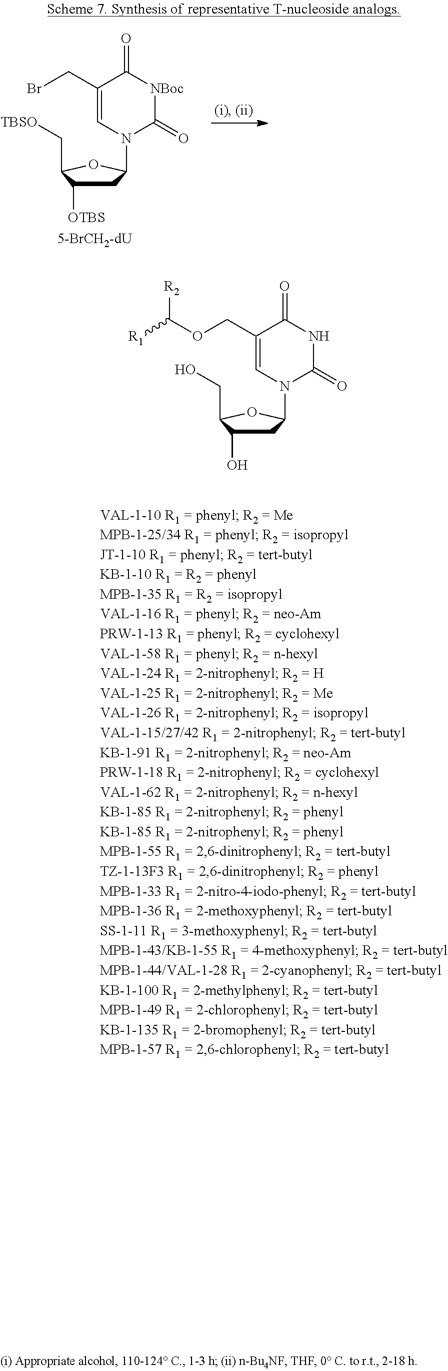

General Procedure—Method A.

N³-tert-Butyloxycarbonyl-5-bromomethyl-3′,5′-bis-O-tert-butyldimethylsilyl-2′-deoxyuridine (5-BrCH₂-dU) and appropriate alcohol (4-20 eq) were heated neat at 110-120° C. for 1-3 hours under argon atmosphere. The mixture was cooled down to room temperature, dissolved in tetrahydrofuran (ca 5 ml), and to this solution chilled at 0° C. tetra-n-butylammonium fluoride trihydrate (TBAF) was added (ca 2.5 eq.). The reaction mixture was stirred for 18 hours while gradually warming up to room temperature. The solvent was removed under reduced pressure and the residue was purified by silica gel (chloroform/methanol=1:0 to 10:1) and then by C8 reverse-phase column chromatography (water/methanol=19:1 to 1:4) to yield the product as a waxy solid.

General Procedure—Method B.

N³-tert-Butyloxycarbonyl-5-bromomethyl-3′,5′-bis-O-tert-butyldimethylsilyl-2′-deoxyuridine (5-BrCH₂-dU) and appropriate alcohol (4-20 eq) were heated neat at 110-120° C. for 0.5-2 hours under argon atmosphere. The mixture was cooled down to room temperature, dissolved in ethyl acetate (ca 5 ml), and silica (0.5-1.0) g was added. The mixture was evaporated, and the solid was applied onto a silica gel chromatography column (hexane/ethyl acetate=15:1 to 2:1). Fractions that were not the starting alcohol were collected, evaporated under reduced pressure, dissolved in tetrahydrofuran (ca 5 mL), and to this solution chilled at 0° C. tetra-n-butylammonium fluoride trihydrate (TBAF) was added (ca 2.5 eq.). The reaction mixture was stirred for 2-3 hours while gradually warming up to room temperature. The solvent was removed under reduced pressure and the residue was purified by silica gel (ethyl acetate/methanol=1:0 to 20:1) to afford product as waxy solid.

### 5-[1-(phenyl)ethoxymethyl]-2′-deoxyuridine (VAL-1-10)

Heating 5-BrCH₂-dU (121 mg, 0.186 mmol) with α-methylbenzyl alcohol (1-phenyl-1-ethanol) (0.228 g, 1.862 mmol) for 1 hour at 114° C. followed by treatment with TBAF (0.303 g, 0.930 mmol) afforded after purification (method A) 20 mg (30%) of product as 1:1 mixture of diastereomers. ¹H NMR (400 MHz, CD₃OD) for diastereomers: δ 7.96 and 7.95 (s, 1H), 7.33 (br. m, 5H), 6.27 (m, 1H), 4.54 (m, 1H), 4.38 (m, 1H), 4.10 (m, 2H), 3.92 (m, 1H), 3.75 (m, 2H) 2.26 (m, 1H), 2.19 (m, 2H), 1.42 (d, 3H, J=6.5). ¹³C NMR (400 MHz, CD₃OD) δ 165.40 (C), 156.37 (C), 139.14 (CH), 128.12 (CH), 127.85 (C), 127.15 (CH), 125.88 (CH), 111.62 (C), 87.54 (CH), 85.12 (CH), 78.04 (CH), 70.79 (CH), 61.40 (CH₂) 39.93 (CH₂), 22.98 (CH₃). HRMS (ESI) for [MH]⁺C₁₈H₂₃N₂O₆ calculated: 363.15506, observed: 363.15516.

### 5-[1-(phenyl)-2-(methyl)-1-propoxymethyl]-2′-deoxyuridine (MPB-1-35/34)

Heating 5-BrCH₂-dU (250 mg, 0.385 mmol) with α-isopropylbenzyl alcohol (2-methyl-1-phenyl-1-propanol) (1.16 g, 7.70 mmol) for 2 hours at 124° C. followed by treatment with TBAF (303 mg, 0.963 mmol) afforded after purification (method A) 92 mg (61%) of product as 1:1 mixture of diastereomers. ¹H NMR (500 MHz, CD₃OD) for diastereomers: δ 7.91 (s, 1H), 7.29 (m, 5H), 6.27 (t, 1H, J=6.7 Hz), 4.39 (m, 1H), 4.05 (m, 3H), 3.93 (m, 1H), 3.75 (m, 2H), 2.27 (m, 1H), 2.18 (m, 1H), 1.91 (m, 1H), 0.99 (m, 3H), 0.72 (m, 3H)¹³C NMR (125 MHz, CD₃OD) for diastereomers: δ 163.63 (C), 150.72 (C), 141.10 and 141.02 (C), 138.94 (CH), 127.80 (CH), 127.23 (CH), 127.14 (CH), 111.56 (C), 87.69 (CH), 87.54 (CH), 85.14 and 85.06 (CH), 70.94 (CH) and 70.87 (CH), 63.39 and 63.20 (CH₂), 61.51 (CH₂), 39.93 (CH₂), 34.67 and 34.61 (CH), 18.09 (CH₃), 17.98 (CH₃). HRMS (ESI) for [MH]⁺C₂₀H₂₇N₂O₆ calculated: 391.18636, observed: 391.18644.

### 5-[1-(phenyl)-2,2-(dimethyl)-1-propoxymethyl]-2′-deoxyuridine (JT-1-10)

Heating 5-BrCH₂-dU (250 mg, 0.385 mmol) with α-tert-butylbenzyl alcohol (2,2-dimethyl-1-phenyl-1-propanol) (1.26 g, 7.70 mmol) for 2 hours at 120° C. followed by treatment with TBAF (607 mg, 1.925 mmol) afforded after purification (method A) 22 mg (21%) of product as 1:1 mixture of diastereomers. ¹H NMR (400 MHz, CD₃OD) for diastereomers: δ 7.93 and 7.92 (2 s, 1H), 7.32 (m, 5H), 6.28 (t, 1H, J=6.7 Hz), 4.42 (m, 1H), 4.06 (m, 3H), 3.95 (m, 1H), 3.77 (m, 2H), 2.30 (m, 1H), 2.19 (m, 1H), 0.91 (m, 9H). ¹³C NMR (100 MHz, CD₃OD) for diastereomers: δ 163.60 (C), 150.68 (C), 139.52 (C), 138.94 and 138.62 (CH), 128.24 and 128.20 (CH), 127.18 (CH), 126.93 (CH), 111.62 (C), 87.66 and 87.56 (CH), 87.58 and 87.51 (CH), 85.14 and 84.99 (CH), 70.99 and 70.89 (CH), 63.79 and 63.53 (CH₂), 61.59 (CH₂), 39.94 and 39.87 (CH₂), 35.11 (C), 25.38 (CH₃). HRMS (ESI) for [MH]⁺C₂₁H₂₉N₂O₆ calculated: 405.20201, observed: 405.20210.

### 5-[(diphenyl)methoxymethyl]-2′-deoxyuridine (KB-1-10)

Heating 5-BrCH₂-dU (250 mg, 0.385 mmol) with diphenylmethanol (1.42 g, 7.70 mmol) for 2.5 hours at 120° C. followed by treatment with TBAF (607 mg, 1.925 mmol) afforded after purification (method A) 6 mg (3%) of product. ¹H NMR (400 MHz, CD₃OD) δ 8.02 (s, 1H), 7.34 (m, 10H), 6.29 (t, 1H, 6.3 Hz), 5.55 (s, 1H), 4.40 (m, 1H), 3.94 (m, 1H), 4.33 (AB dd, 1H, J=12.1, 1.0), 4.28 (AB dd, 1H, J=12.1, 1.0), 3.79 (AB dd, 1H, J=11.8, 3.1), 3.73 (AB dd, 1H, J=11.8, 3.1), 2.28 (m, 1H), 2.20 (m, 1H). ¹³C NMR (100 MHz, CD₃OD) δ 163.80 (C), 150.69 (C), 142.19 (C), 139.18 (CH), 127.96 (CH), 127.09 (CH), 126.72 (CH), 111.24 (C), 86.56 (CH), 85.14 (CH), 83.39 (CH), 70.85 (CH), 63.47 (CH₂), 61.52 (CH₂), 39.93 (CH₂). HRMS (ESI) for [MH]⁺C₂₃H₂₅N₂O₆ calculated: 425.17071, observed: 425.17082.

### 5-[1-(isopropyl)-2-(methyl)-1-propoxymethyl]-2′-deoxyuridine (MPB-1-35)

Heating 5-BrCH₂-dU (250 mg, 0.385 mmol) with 2,4-dimethyl-3-pentanol (0.894 g, 7.70 mmol) for 2 hours at 124° C. followed by treatment with TBAF (303 mg, 0.963 mmol) afforded after purification (method A) 33 mg (24%) of product. ¹H NMR (400 MHz, CD₃OD) δ 8.03 (s, 1H), 6.30 (t, 1H, J=6.7 Hz), 4.40 (m, 1H), 4.34 (AB d, 1H, J=11.7), 4.29 (AB d, 1H, J=11.7), 3.93 (q, 1H, J=3.5), 3.76 (AB dd, 1H, J=11.9, 3.6), 3.72 (AB dd, 2H, J=11.9, 3.7), 2.87 (t, 1H, J=5.7), 2.29 (m, 1H), 2.23 (m, 1H), 1.84 (m, 2H), 0.94 (m, 12H). ¹³C NMR (100 MHz, CD₃OD) δ 163.64 (C), 150.76 (C), 139.02 (CH), 109.98 (C), 90.32 (CH), 87.57 (CH), 85.10 (CH), 70.93 (CH), 67.12 (CH₂), 61.54 (CH₂), 39.93 (CH₂), 30.57 (CH), 19.30 (CH₃), 16.73 (CH₃). HRMS (ESI) for [MH]⁺C₁₇H₂₉N₂O₆ calculated: 357.20201, observed: 357.20210.

### 5-[1-(phenyl)-3,3-(dimethyl)-1-butoxymethyl]-2′-deoxyuridine (VAL-1-16)

Heating 5-BrCH₂-dU (44 mg, 0.068 mmol) with α-neo-pentylbenzyl alcohol (3,3-dimethyl-1-phenyl-1-butanol) (41 mg, 0.239 mmol) for 2.5 hours at 110° C. followed by treatment with TBAF (53 mg, 0.170 mmol) afforded after purification (method A) 12 mg (43%) of product as 1:1 mixture of diastereomers. ¹H NMR (400 MHz, CD₃OD) for diastereomers: δ 7.97 and 7.95 (s, 1H), 7.34 (m, 5H), 6.29 (m, 1H), 4.51 (m, 1H), 4.41 (m, 1H), 4.05 (m, 2H), 3.94 (m, 1H), 3.78 (m, 2H), 2.28 (m, 1H), 2.21 (m, 1H), 1.82 (m, 1H), 1.44 (m, 1H), 0.99 and 0.98 (s, 9H). ¹³C NMR (100 MHz, CD₃OD) for diastereomersδ 163.60 (C), 150.63 (C), 143.72 (C), 139.23 and 139.06 (CH), 128.13 (CH), 127.00 (CH), 126.28 and 126.21 (CH), 111.47 (C), 86.51 (CH), 85.09 and 84.93 (CH), 80.08 and 79.97 (CH), 70.85 (CH), 62.71 and 62.52 (CH₂), 61.52 and 61.49 (CH₂), 51.94 and 51.82 (CH₂), 40.01 (CH₂), 30.01 (C), 29.32 (CH₃). HRMS (ESI) for [MH]⁺C₂₂H₃₁N₂O₆ calculated: 419.21766, observed: 419.21780.

### 5-[1-(phenyl)-1-(cyclohexyl)methoxymethyl]-2′-deoxyundine (PRW-1-13)

Heating 5-BrCH₂-dU (97 mg, 0.149 mmol) with α-cyclohexylbenzyl alcohol (550 mg, 2.890 mmol) for 2.5 hours at 132° C. followed by purification of bis- and mono-TBS products with subsequent treatment with TBAF (103 mg, 0.326 mmol) afforded after purification (method B) 22 mg (34%) of product as 1:1 mixture of diastereomers. ¹H NMR (400 MHz, CD₃OD) for diastereomers: δ 7.90 (s, 1H), 7.28 (m, 5H), 6.26 (m, 1H), 4.39 (m, 1H), 4.06 (m, 3H), 3.93 (m, 1H), 3.75 (m, 2H), 2.28 (m, 1H), 2.17 (m, 1H), 2.04 (m, 1H), 1.73 (m, 1H), 1.60 (m, 3H), 1.05 (m, 6H). ¹³C NMR (100 MHz, CD₃OD) for diastereomers: δ 165.00 and 164.98 (C), 154.08 (C), 142.40 (C), 140.29 and 140.26 (CH), 129.15 (CH), 128.64 and 128.57 (CH), 128.48 and 128.46 (CH), 112.92 (C), 88.94 and 88.90 (CH), 88.14 (CH), 86.54 and 86.47 (CH), 72.36 and 72.27 (CH), 64.66 and 64.45 (CH₂), 62.94 and 62.90 (CH₂), 45.71 and 45.70 (CH), 41.32 (CH₂), 30.62 and 30.41 (CH₂), 27.65 (CH₂), 27.17 and 27.12 (CH₂). HRMS (ESI) for [MNa]⁺C₂₃H₃₀N₂O₆Na calculated: 453.19961, observed: 453.19977.

### 5-[1-(phenyl)-1-hexyloxymethyl]-2′-deoxyuridine (VAL-1-58)

Heating 5-BrCH₂-dU (103 mg, 0.158 mmol) with α-n-hexylbenzyl alcohol (1-phenyl-1-hexanol, 376 mg, 1.580 mmol) for 2 hours at 116° C. followed by purification of bis- and mono-TBS products with subsequent treatment with TBAF (161 mg, 0.457 mmol) afforded after purification (method B) 18 mg (27%) of product as 1:1 mixture of diastereomers. ¹H NMR (400 MHz, CD₃OD) for diastereomers: δ 7.96 (s, 1H), 7.31 (m, 5H), 6.28 (m, 1H), 4.42 (m, 1H), 4.36 (m, 1H), 4.11 (m, 2H), 3.95 (m, 1H), 3.80 (AB d, 1H, J=12.0 Hz), 3.75 (AB d, 1H, J=12.0 Hz), 2.28 (m, 1H), 2.21 (m, 1H), 1.82 (m, 1H), 1.62 (m, 1H), 1.28 (m, 8H), 0.89 (m, 3H). ¹³C NMR (100 MHz, CD₃OD) for diastereomers: δ 163.65 (C), 150.69 (C), 142.52 (C), 139.21 (CH), 120.05 (CH), 127.17 (CH), 126.49 and 126.43 (CH), 111.38 (C), 87.56 (CH), 85.16 and 85.09 (CH), 82.11 and 82.00 (CH), 70.91 and 70.85 (CH), 63.11 and 62.91 (CH₂), 61.50 (CH₂), 39.99 (CH₂), 37.89 and 37.86 (CH₂), 31.58 (CH₂), 28.89 (CH₂), 25.44 and 25.39 (CH₂), 22.27 (CH₂), 13.04 (CH₃).

### 5-[1-(2-nitrobenzyl)oxymethyl]-2′-deoxyuridine (VAL-1-24)

Heating 5-BrCH₂-dU (220 mg, 0.339 mmol) with 2-nitrobenzyl alcohol (233 mg, 1.524 mmol) for 20 minutes at 90-105° C. afforded after purification (method A) 21 mg (16%) of product. ¹H NMR (400 MHz, CD₃OD): δ 8.12 (s, 1H), 8.04 (dd, 1H, J=8.2, 1.1 Hz), 7.84 (d, 1H, J=7.0 Hz), 7.71 (dt, 1H, J=7.6, 1.1 Hz), 7.52 (m, 1H), 6.30 (t, 1H, J=6.7 Hz), 4.93 (s, 2H), 4.43 (m, 1H), 4.39 (AB d, 1H, J=11.8 Hz), 4.34 (AB d, 1H, J=11.8 Hz), 3.95 (q, 1H, J=3.4 Hz), 3.82 (AB d, 1H, J=12.0, 3.8 Hz), 3.75 (AB d, 1H, J=12.0, 3.3 Hz), 2.29 (m, 2H).

### 5-[1-(2-nitrophenyl)ethoxymethyl]-2′-deoxyuridine (VAL-1-25)

Heating 5-BrCH₂-dU (152 mg, 0.234 mmol) with α-methyl-2-nitrobenzyl alcohol (1-(2-nitro)phenyl-1-ethanol) (176 mg, 1.053 mmol) for 1 hour at 104° C. afforded after purification (method A) 14 mg (15%) of product as 1:1 mixture of diastereomers. ¹H NMR (400 MHz, CD₃OD) for diastereomers: δ 8.01 and 8.00 (2 s, 1H), 7.93 (m, 1H), 7.85 (m, 1H), 7.72 (t, 1H, J=7.5 Hz), 6.27 (m, 1H), 5.09 (m, 1H), 4.42 (m, 1H), 4.11 (m, 2H), 3.94 (m, 1H), 3.78 (m, 2H), 2.26 (m, 2H), 1.52 and 1.52 (2 d, 3H, J=6.3 Hz). ¹³C NMR (100 MHz, CD₃OD) for diastereomers: δ 163.63 (C), 150.65 (C), 148.48 (C), 139.71 and 139.68 (CH), 138.99 (C), 133.27 and 133.24 (CH), 128.02 (CH), 127.81 and 127.78 (CH), 123.74 and 123.70 (CH), 110.92 and 110.84 (C), 87.58 and 87.56 (CH), 85.13 and 85.12 (CH), 73.19 and 73.01 (CH), 70.81 and 70.78 (CH), 63.60 and 63.46 (CH₂), 61.40 and 61.38 (CH₂), 39.99 (CH₂), 22.47 (CH₃).

### 5-[1-(2-nitrophenyl)ethoxymethyl]-2′-deoxyuridine (VAL-1-26)

Heating 5-BrCH₂-dU (175 mg, 0.270 mmol) with α-isopropyl-2-nitrobenzyl alcohol (1-(2-nitro)phenyl-2-methyl-1-propanol) (400 mg, 2.050 mmol) for 1 hour at 105-114° C. afforded after purification (method A) 16 mg (14%) of product as 1:1 mixture of diastereomers. ¹H NMR (400 MHz, CD₃OD) for diastereomers: δ 8.01 and 7.98 (2 s, 1H), 7.90 (d, 1H, J=8.5 Hz), 7.77 (m, 1H), 7.51 (m, 1H), 6.27 (m, 1H), 4.78 (m, 1H), 4.41 (m, 1H), 4.13 (m, 2H), 3.94 (m, 1H), 3.77 (m, 2H), 2.25 (m, 2H), 1.96 (m, 1H), 0.97 and 0.96 (2 d, 3H, J=6.7 Hz), 0.88 and 0.86 (2 d, 3H, J=7.0 Hz). ¹³C NMR (100 MHz, CD₃OD) for diastereomers: δ 163.63 and 163.56 (C), 150.66 and 150.64 (C), 149.52 (C), 139.66 and 139.50 (CH), 136.52 and 136.51 (C), 132.56 and 132.54 (CH), 129.01 and 128.95 (CH), 128.05 (CH), 123.71 and 123.65 (CH), 111.05 and 110.86 (C), 87.57 (CH), 85.08 and 85.07 (CH), 81.08 and 80.82 (CH), 70.90 (CH), 64.23 and 63.96 (CH₂), 61.48 and 61.45 (CH₂), 39.95 and 39.90 (CH₂), 34.67 (CH), 18.31 and 18.26 (CH₃), 16.64 and 16.57 (CH₃). HRMS (ESI) for [MNa]⁺ C₂₀H₂₅N₃O₈Na calculated: 458.15339, observed: 458.15342; for [M-H]C₂₀H₂₄N₃O₈ calculated: 434.15689, observed: 434.15669.

### 5-[1-(2-nitrophenyl)-2,2-(dimethyl)propoxymethyl]-2′-deoxyuridine (VAL-1-15/27/42)

Heating 5-BrCH₂-dU (250 mg, 0.385 mmol) with α-tert-butyl-2-nitrobenzyl alcohol (2,2-dimethyl-1-(2-nitro)phenyl-1-propanol) (1.61 g, 7.70 mmol) for 2 hours at 120° C. followed by treatment with TBAF (607 mg, 1.925 mmol) afforded after purification (method A) 33 mg (19%) of product as 1:1 mixture of diastereomers. ¹H NMR (400 MHz, CD₃OD) for diastereomers: δ 8.01 and 7.99 (2 s, 1H), 7.81 (m, 2H), 7.68 (m, 1H), 7.51 (m, 1H), 6.28 (t, 1H, J=6.9 Hz), 4.98 (s, 1H), 4.42 (m, 1H), 4.20 (m, 2H), 3.94 (m, 1H), 3.76 (m, 2H), 2.26 (m, 2H), 0.85 and 0.84 (2 s, 9H). ¹³C NMR (100 MHz, CD₃OD) for diastereomers: δ 163.60 and 163.55 (C), 150.89 and 150.75 (C), 150.70 (C), 139.80 and 139.41 (CH), 133.81 (C), 131.76 and 131.74 (CH), 129.91 and 129.82 (CH), 128.14 (CH), 123.56 and 123.43 (CH), 111.01 and 110.74 (C), 87.55 (CH), 85.13 and 85.04 (CH), 81.76 and 81.04 (CH), 70.98 and 70.95 (CH), 64.49 and 64.18 (CH₂), 61.52 and 61.46 (CH₂), 39.86 and 39.78 (CH₂), 36.12 and 36.02 (C), 24.84 and 24.82 (CH₃). HRMS (ESI) for [MH]⁺ O₂₁H₂₈N₃O₈ calculated: 450.18709, observed: 450.18708; for [M-H]⁻ C₂₁H₂₆N₃O₈ calculated: 448.17254, observed: 448.17258.

### 5-[1-(2-nitrophenyl)-3,3-(dimethyl)butoxymethyl]-2′-deoxyuridine (KB-1-91)

Heating 5-BrCH₂-dU (210 mg, 0.323 mmol) with α-neo-pentyl-2-nitrobenzyl alcohol (3,3-dimethyl-1-(2-nitro)phenyl-1-butanol) (480 mg, 2.152 mmol) for 2 hours at 120° C. followed by treatment with TBAF (607 mg, 1.925 mmol) afforded after purification 18 mg (12%) of product as 1:1 mixture of diastereomers. ¹H NMR (400 MHz, CD₃OD) for diastereomers: δ 8.02 and 8.00 (2 s, 1H), 7.90 (d, 1H, J=8.2 Hz), 7.82 (m, 1H) 7.70 (t, 1H, J=7.6 Hz), 7.48 (m, 1H), 6.27 (t, 1H, J=6.9 Hz), 5.14 (m, 1H), 4.42 (m, 1H), 4.05 (m, 2H), 3.94 (m, 1H), 3.78 (m, 2H), 2.28 (m, 1H), 2.19 (m, 1H), 1.71 (m, 1H), 1.53 (m, 1H), 1.05 and 1.04 (2 s, 9H). ¹³C NMR (100 MHz, CD₃OD) for diastereomers: δ 163.55 and 163.51 (C), 150.64 and 150.60 (C), 148.26 and 148.22 (C), 139.90 and 139.66 (CH), 139.16 (C), 137.73 (CH), 133.16 and 133.11 (CH), 128.29 and 127.82 (CH), 123.73 and 123.67 (CH), 111.10 and 110.88 (C), 87.67 and 87.58 (CH), 85.07 and 84.97 (CH), 75.02 and 74.74 (CH), 70.89 (CH), 63.25 and 63.13 (CH₂), 61.46 (CH₂), 51.05 and 51.01 (CH₂), 40.05 and 39.93 (CH₂), 30.42 and 30.39 (C), 29.40 and 29.38 (CH₃). HRMS (ESI) for [MNa]⁺ C₂₂H₂₉N₃O₈Na calculated: 486.18430, observed: 486.18520.

### 5-[1-(2-nitro)phenyl-1-(cyclohexyl)methoxymethyl]-2′-deoxyuridine (PRW-1-18)

Heating 5-BrCH₂-dU (150 mg, 0.231 mmol) with α-cyclohexyl-2-nitrobenzyl alcohol (440 mg, 1.880 mmol) for 2.5 hours at 116° C. followed by purification of bis- and mono-TBS products with subsequent treatment with TBAF (73 mg, 0.231 mmol) afforded after purification (method B) 28 mg (25%) of product as 1:1 mixture of diastereomers. ¹H NMR (400 MHz, CD₃OD) for diastereomers: δ 7.99 and 7.96 (2 s, 1H), 7.89 (d, J=8.1 Hz, 1H), 7.71 (m, 2H), 7.50 (d, J=7.6 Hz, 1H), 6.27 (t, J=6.6 Hz, 1H), 4.78 (m, 1H), 4.42 (m, 1H), 4.12 (m, 3H), 3.94 (m, 1H), 3.77 (m, 2H), 2.28 (m, 1H), 2.22 (m, 1H), 1.87 (m, 1H), 1.70 (m, 2H), 1.30 (m, 4H), 1.17 (m, 4H). ¹³C NMR (100 MHz, CD₃OD) for diastereomers δ: 163.57 (C), 150.65 (C), 149.62 and 149.53 (C), 139.61 and 139.51 (CH), 136.21 (C), 132.48 (CH), 129.11 and 129.05 (CH), 128.02 (CH), 123.65 and 123.59 (CH), 111.02 and 110.86 (C), 87.58 (CH), 85.07 (CH), 80.64 and 80.23 (CH), 70.94 and 70.91 (CH), 64.15 and 63.96 (CH₂), 61.50 (CH₂), 44.50 and 44.47 (CH), 39.92 and 39.89 (CH₂), 29.22 and 29.15 (CH₂), 28.05 and 27.98 (CH₂), 26.09 (CH₂), 25.96 and 25.94 (CH₂), 25.78 (CH₂). HRMS (ESI) for [MH]⁺C₂₃H₃₀N₃O₈ calculated: 476.20274, observed: 476.20292; for [MNa]⁺C₂₃H₂₉N₃O₈Na calculated: 498.18469, observed: 498.18486.

### 5-[1-(2-nitro)phenyl-1-hepthoxymethyl]-2′-deoxyuridine (VAL-1-62)

Heating 5-BrCH₂-d U (97 mg, 0.149 mmol) with α-hexyl-2-nitrobenzyl alcohol (1-(2-nitro)phenyl-1-heptanol) (142 mg, 0.597 mmol) for 15 minutes at 112-114° C. followed by purification of bis- and mono-TBS products with subsequent treatment with TBAF (48 mg, 0.150 mmol) afforded after purification (method B) 18 mg (25%) of product as 1:1 mixture of diastereomers. ¹H NMR (400 MHz, CD₃OD) for diastereomers: δ 8.02 and 7.98 (2 s, 1H), 7.92 (d, J=8.4 Hz, 1H), 7.80 (d, J=8.0 Hz, 1H), 7.69 (m, 1H), 7.48 (m, 1H), 6.25 (m, 1H), 4.40 (m, 1H), 4.09 (m, 3H), 3.92 (m, 1H), 3.74 (m, 2H), 2.24 (m, 2H), 1.70 (m, 2H), 1.47 (m, 4H), 0.92 (m, 7H). ¹³C NMR (100 MHz, CD₃OD) for diastereomers: δ 165.09 (C), 150.00 and 149.98 (C), 141.34 and 141.22 (CH), 139.82 (C), 134.50 (CH), 129.60 and 129.56 (CH), 129.34 (CH), 125.23 (C), 125.17 (CH), 112.38 (C), 89.03 (CH), 86.50 (CH), 78.19 and 77.46 (CH), 72.32 and 72.77 (CH), 65.29 and 65.11 (CH₂), 62.86 (CH₂), 41.47 and 41.40 (CH₂), 39.16 (CH₂), 33.10 and 32.97 (CH₂), 30.81 and 30.49 (CH₂), 27.08 (CH₂), 23.77 and 23.72 (CH₂), 14.45 (CH₃).

### 5-[{(2-nitrophenyl)phenyl}methoxymethyl]-2′-deoxyundine (KB-1-85)

Heating 5-BrCH₂-dU (210 mg, 0.385 mmol) with α-phenyl-2-nitrobenzyl alcohol (phenyl(2-nitrophenyl)methanol) (361 mg, 1.576 mmol) for 2.5 hours at 110-117° C. followed by purification of bis- and mono-TBS products with subsequent treatment with TBAF (73 mg, 0.231 mmol) afforded after purification (method A) 12 mg (7%) of product as 1:1 mixture of diastereomers. ¹H NMR (400 MHz, CD₃OD) for diastereomers: δ 8.04 and 8.00 (2 s, 1H), 7.88 (m, 1H), 7.68 (m, 2H), 7.52 (m, 1H), 7.34 (m, 5H), 6.28 (m, 1H), 6.18 and 6.17 (2 s, 1H), 4.42 (m, 1H), 4.30 (m, 3H), 3.94 (m, 1H), 3.78 (m, 2H), 2.28 (m, 2H). ¹³C NMR (100 MHz, CD₃OD) for diastereomers: δ 163.71 (C), 159.43 (C), 158.87 (C), 150.64 (C), 140.03 and 139.74 (CH), 136.21 and 136.18 (C), 132.60 (CH), 128.13 (CH), 128.11 (CH), 128.09 (CH), 127.58 (CH), 123.92 (CH), 123.87 (CH), 110.71 (C), 87.59 (CH), 85.13 (CH), 78.29 and 78.16 (CH), 70.82 (CH), 64.01 and 63.91 (CH₂), 61.48 (CH₂), 39.95 (CH₂). HRMS (ES⁺ TOF) for [MNa]⁺C₂₃H₂₉N₃O₈Na calculated: 492.13830, observed: 492.13830.

### 5-[1-(2,6-dinitrophenyl)-2,2-(dimethyl)propoxymethyl]-2′-deoxyuridine (MPB-1-55)

Heating 5-BrCH₂-dU (259 mg, 0.399 mmol) with α-tert-butyl-2,6-dinitrobenzyl alcohol (2,2-dimethyl-1-(2,6-dinitro)phenyl-1-propanol) (342 g, 1.345 mmol) for 10 minutes at 105° C. followed by treatment with TBAF (314 mg, 0.997 mmol) afforded after purification (method A) 23 mg (12%) of product as 1:1 mixture of diastereomers. ¹H NMR (400 MHz, CD₃OD) for diastereomers: δ 8.05 (m, 1H), 8.01 and 7.86 (2 s, 1H), 7.74 (m, 2H), 6.39 and 6.34 (2 t, J=6.7 Hz, 1H), 5.20 and 5.19 (2 s, 1H), 4.44 (m, 1H), 4.25 (m, 2H), 3.94 (m, 1H), 3.74 (m, 2H), 2.33 (m, 2H), 0.87 (s, 9H). ¹³C NMR (100 MHz, CD₃OD) for diastereomers (NOTE: due to the presence of two ortho-substituents, there is, apparently, restricted rotation of the 2,6-dinitrophenyl group around its 1-C-4-C axis, which thereby makes 2-CNO₂ non-equivalent to 6-CNO₂, and accordingly, 3-CH non-equivalent to 5-CH): δ 163.62 and 163.55 (C), 150.89 and 150.74 (C), 151.25 (C), 151.13 (C), 140.62 and 139.45 (CH), 130.13 and 130.04 (CH), 128.06 and 128.01 (CH), 126.26 and 126.16 (CH), 125.47 and 125.32 (C), 109.97 and 109.84 (C), 87.55 and 87.35 (CH), 85.05 and 84.56 (CH), 82.85 and 82.05 (CH), 71.07 and 70.96 (CH), 66.23 and 65.85 (CH₂), 61.69 (CH₂), 39.82 and 39.50 (CH₂), 37.88 and 37.83 (C), 25.72 (CH₃). HRMS (ESI) for [MH]⁺C₂₁H₂₇N₄O₁₀ calculated: 495.17217, observed: 495.17218; for [M-H]⁻ C₂₁H₂₆N₄O₁₀ calculated: 493.15762, observed: 493.15754.

### 5-[{(2,6-dinitrophenyl)phenyl}methoxymethyl]-2′-deoxyuridine (TZ-1-13F3)

Heating 5-BrCH₂-dU (87 mg, 0.134 mmol) with α-phenyl-2,6-dinitrobenzyl alcohol (phenyl(2,6-dinitrophenyl)methanol) (81 mg, 0.296 mmol) for 15 minutes at 105-110° C. followed by purification of bis- and mono-TBS products with subsequent treatment with TBAF (75 mg, 0.238 mmol) afforded after purification (method A) 3 mg (4%) of product as 1:1 mixture of diastereomers. ¹H NMR (400 MHz, CD₃OD) for diastereomers: δ 8.10 and 8.09 (2 d, J=8.1 Hz, 1H), 7.95 and 7.89 (2 s, 1H), 7.81 (m, 1H), 7.27 (m, 5H), 6.27 (m, 1H), 6.23 and 6.19 (2 s, 1H), 4.42 (m, 1H), 4.30 (m, 3H), 3.94 (m, 1H), 3.78 (m, 2H), 2.27 (m, 2H). ¹³C NMR (100 MHz, CD₃OD) for diastereomers (NOTE: due to the presence of two ortho-substituents, there is, apparently, restricted rotation of the 2,6-dinitrophenyl group around its 1-C-4-C axis, which thereby makes 2-CNO₂ non-equivalent to 6-CNO₂, and accordingly, 3-CH non-equivalent to 5-CH): δ 163.71 (C), 159.43 (C), 158.87 (C), 150.64 (C), 140.03 and 139.74 (CH), 136.21 (C), 136.18 (C), 134.60 (CH), 132.60 (CH), 128.13 (CH), 128.11 (CH), 128.09 (CH), 127.58 (CH), 123.92 (CH), 123.87 (CH), 110.71 (C), 87.59 (CH), 85.13 (CH), 78.29 and 78.16 (CH), 70.82 (CH), 64.01 and 63.91 (CH₂), 61.48 (CH₂), 39.95 (CH₂). HRMS (ESI) for [MNa]⁺C₂₃H₂₂N₄O₁₀Na calculated: 537.11281, observed: 537.12301.

### 5-[1-(4-iodo-2-nitrophenyl)-2,2-(dimethyl)propoxymethyl]-2′-deoxyuridine (MPB-1-33)

Heating 5-BrCH₂-dU (400 mg, 0.616 mmol) with α-tert-butyl-4-iodo-2-nitrobenzyl alcohol (2,2-dimethyl-1-(4-iodo-2-nitro)phenyl-1-propanol) (717 mg, 2.140 mmol) for 2 hours at 120° C. followed by treatment with TBAF (607 mg, 1.925 mmol) afforded after purification (method A) 163 mg (28%) of product as 1:1 mixture of diastereomers. ¹H NMR (400 MHz, CD₃OD) for diastereomers: δ 8.17 (t, J=1.7 Hz, 1H), 8.03 and 8.01 (2 s, 1H), 8.02 (dd, J=8.4, 1.7 Hz, 1H), 7.54 (d, J=8.4 Hz, 1H), 6.27 (m, 1H), 4.87 (s, 1H), 4.41 (m, 1H), 4.19 (m, 2H), 3.94 (m, 1H), 3.76 (m, 2H), 2.25 (m, 2H), 0.85 and 0.83 (2 s, 9H). ¹³C NMR (100 MHz, CD₃OD) for diastereomers: δ 163.60 and 163.55 (C), 150.69 and 150.61 (C), 150.48 (C), 140.01 and 139.71 (CH), 134.73 and 134.69 (CH), 134.52 (C), 130.36 and 130.26 (CH), 126.71 and 126.61 (CH), 122.79 (C), 110.82 and 110.57 (C), 87.61 (CH), 85.20 and 85.10 (CH), 81.61 and 81.01 (CH), 70.97 and 70.90 (CH), 64.57 and 64.30 (CH₂), 61.48 (CH₂), 39.93 and 39.86 (CH₂), 36.24 and 36.14 (C), 24.78 (CH₃). HRMS (ESI) for [MH]⁺C₂₁H₂₇IN₃O₈ calculated: 576.08428, observed: 576.08383; for [MNa]⁺C₂₁H₂₆IN₃O₈Na calculated: 598.06623, observed: 598.06581.

### 5-[1-(2-methoxyphenyl)-2,2-(dimethyl)propoxymethyl]-2′-deoxyuridine (MPB-1-36)

Heating 5-BrCH₂-dU (250 mg, 0.385 mmol) with α-tert-butyl-2-methoxybenzyl alcohol (3,3-dimethyl-1-(2-methoxy)phenyl-1-propanol) (625 mg, 3.460 mmol) for 2.5 hours at 114-128° C. followed by treatment with TBAF (303 mg, 0.963 mmol) afforded after purification (method A) 94 mg (56%) of product as 1:1 mixture of diastereomers. ¹H NMR (400 MHz, CD₃OD) for diastereomers: δ 7.82 and 7.81 (2 s, 1H), 7.33 (d, 1H, J=7.8 Hz), 7.21 (m, 1H), 6.90 (m, 2H), 6.25 (m, 1H), 4.62 and 4.61 (2 s, 1H), 4.40 (m, 1H), 4.01 (m, 3H), 3.79 (s, 3H), 3.73 (m, 2H), 2.28 (m, 1H), 2.17 (m, 1H), 0.89 and 0.88 (2 s, 9H). ¹³C NMR (100 MHz, CD₃OD) for diastereomers: δ 163.53 (C), 157.92 (C), 150.71 (C), 138.62 and 138.27 (CH), 128.33 (CH), 127.93 (CH), 127.78 (C), 119.65 (CH), 111.82 and 111.56 (C), 109.90 (CH), 87.51 (CH), 85.10 (CH), 81.13 and 80.05 (CH), 71.02 (CH), 63.69 and 63.46 (CH₂), 61.62 (CH₂), 54.33 (CH₃), 39.86 and 39.78 (CH₂), 35.78 and 35.74 (C), 25.21 (CH₃). HRMS (ESI) for [MH]⁺C₂₂H₃₁N₂O₇ calculated: 435.21258 observed: 435.21261; for [MNa]⁺C₂₂H₃₀N₂O₇Na calculated: 457.19452 observed: 457.19451.

### 5-[1-(3-methoxyphenyl)-2,2-(dimethyl)propoxymethyl]-2′-deoxyundine (SS-1-11)

Heating 5-BrCH₂-dU (150 mg, 0.231 mmol) with α-tert-butyl-3-methoxybenzyl alcohol (3,3-dimethyl-1-(3-methoxy)phenyl-1-propanol) (200 mg, 1.030 mmol) for 2 hours at 120° C. followed by treatment with TBAF (182 mg, 0.578 mmol) afforded after purification (method A) 5 mg (5%) of product as 1:1 mixture of diastereomers. ¹H NMR (400 MHz, CD₃OD) for diastereomers: δ 7.91 and 7.91 (2 s, 1H), 7.22 (m, 1H), 6.85 (m, 3H), 6.27 (m, 1H), 4.42 (2 s, 1H), 4.09 (m, 3H), 3.95 (m, 1H), 3.81 and 3.81 (2 s, 3H), 3.77 (m, 2H), 2.27 (m, 2H), 0.92 (s, 9H). ¹³C NMR (100 MHz, CD₃OD) for diastereomers: δ 159.22 and 159.19 (C), 150.73 and 150.68 (C), 141.36 and 141.17 (C), 138.86 and 138.72 (CH), 128.12 and 128.10 (CH), 120.77 and 120.67 (CH), 115.38 and 115.31 (C), 113.72 and 113.64 (CH), 112.31 (CH), 111.82 and 111.56 (C), 89.60 and 89.45 (CH), 87.59 and 87.52 (CH), 85.15 and 84.98 (CH), 71.00 and 70.90 (CH), 64.07 and 63.66 (CH₂), 61.58 and 61.51 (CH₂), 54.20 (CH₃), 39.94 and 39.89 (CH₂), 35.10 and 35.08 (C), 25.45 and 25.43 (CH₃). HRMS (ES⁺ TOF) for [MH]⁺C₂₂H₃₁N₂O₇ calculated: 435.21258 observed: 435.21259; for [MNa]⁺C₂₂H₃₀N₂O₇Na calculated: 457.19452 observed: 457.19450; for [M-H]⁻ C₂₂H₂₉N₂O₇ calculated: 433.19802 observed: 433.19809.

### 5-[1-(4-methoxyphenyl)-2,2-(dimethyl)propoxymethyl]-2′-deoxyuridine (MPB-1-43/KB-1-55)

Heating 5-BrCH₂-dU (346 mg, 0.539 mmol) with α-tert-butyl-4-methoxybenzyl alcohol (3,3-dimethyl-1-(4-methoxy)phenyl-1-propanol) (620 mg, 2.150 mmol) for 2.5 hours at 120° C. followed by treatment with TBAF (870 mg, 2.762 mmol) afforded after purification (method A) 11 mg (5%) of product as 1:1 mixture of diastereomers. ¹H NMR (400 MHz, CD₃OD) for diastereomers: δ 7.89 and 7.87 (s, 1H), 7.19 (d, J=8.6 Hz, 2H), 6.87 (m, 2H), 6.27 (m, 1H), 4.40 (m, 1H), 4.05 (m, 3H), 3.94 (m, 1H), 3.78 and 3.78 (2 s, 3H), 3.76 (m, 2H), 2.28 (m, 1H), 2.18 (m, 1H), 0.88 (s, 9H). ¹³C NMR (100 MHz, CD₃OD) for diastereomers δ 163.75 (C), 159.05 (C), 150.80 (C), 138.62 and 138.51 (CH), 131.39 and 131.32 (C), 129.24 and 129.22 (CH), 112.62 (CH), 111.78 and 111.72 (C), 89.28 and 89.16 (CH), 87.57 and 87.49 (CH), 85.15 and 85.00 (CH), 71.01 and 70.91 (CH), 63.62 and 63.38 (CH₂), 61.61 and 61.57 (CH₂), 54.28 (CH₃), 39.93 and 39.83 (CH₂), 35.20 (C), 25.38 (CH₃). HRMS (ES⁺ TOF) [MNa]⁺ C₂₂H₃₀N₂O₇Na calculated: 457.19452 observed: 457.19490.

### 5-[1-(2-cyanophenyl)-2,2-(dimethyl)propoxymethyl]-2′-deoxyuridine (MPB-1-44/VAL-1-28)

Heating 5-BrCH₂-dU (114 mg, 0.176 mmol) with α-tert-butyl-2-cyanobenzyl alcohol (2,2-dimethyl-1-(2-cyano)phenyl-1-propanol) (1.61 g, 7.70 mmol) for 2 hours at 120° C. followed by treatment with TBAF (607 mg, 1.925 mmol) afforded after purification (method A) 14 mg (18%) of product as 1:1 mixture of diastereomers. ¹H NMR (400 MHz, CD₃OD) for diastereomers: δ 7.99 and 7.98 (2 s, 1H), 7.89 (d, J=7.7 Hz, 1H), 7.54 (m, 3H), 6.32 (m, 1H), 5.33 (s, 1H), 4.38 (m, 3H), 3.91 (m, 1H), 3.68 (m, 2H), 2.24 (m, 2H), 1.01 (s, 9H). ¹³C NMR (100 MHz, CD₃OD) for diastereomers: δ 164.07 (C), 150.86 (C), 145.06 (C), 137.80 (CH), 131.33 (CH), 130.46 (C), 128.57 (CH), 123.06 and 122.93 (CH), 115.25 (CH), 112.98 (C), 91.70 (CH), 87.56 and 87.52 (CH), 85.11 and 85.02 (CH), 71.00 and 70.92 (CH), 61.55 (CH₂), 58.09 (CH₂), 39.83 (CH₂), 35.58 (C), 24.29 (CH₃). HRMS (ES⁺ TOF) for [MH]⁺C₂₂H₂₈N₃O₆ calculated: 430.19870, observed: 430.19700; for [MNa]⁺C₂₂H₂₇N₃O₆Na calculated: 452.17970, observed: 452.18040.

### 5-[1-(2-methyl)phenyl-2,2-(dimethyl)propoxymethyl]-2′-deoxyuridine (KB-1-100)

Heating 5-BrCH₂-dU (125 mg, 0.195 mmol) with α-tert-butyl-2-methylbenzyl alcohol (3,3-dimethyl-1-(2-methyl)phenyl-1-propanol) (174 mg, 0.776 mmol) for 1 hour at 112° C. Purification (method A) afforded 8 mg (10%) of product as 1:1 mixture of diastereomers. ¹H NMR (400 MHz, CD₃OD) for diastereomers: δ 7.89 and 7.88 (2 s, 1H), 7.41 (m, 1H), 7.16 (m, 3H), 6.28 (m, 1H), 4.46 (s, 1H), 4.41 (m, 1H), 4.03 and 4.01 (2 s, 3H), 3.95 (m, 1H), 3.76 (m, 2H), 2.38 and 2.37 (2 s, 3H), 2.30 (m, 1H), 2.19 (m, 1H), 0.99 (s, 9H). ¹³C NMR (100 MHz, CD₃OD) for diastereomers: δ 163.57 (C), 150.67 (C), 138.45 and 138.39 (CH), 136.84 (C), 136.74 (C), 129.76 (CH), 127.76 and 127.72 (CH), 126.71 and 126.66 (CH), 124.97 and 124.93 (CH), 111.82 (C), 87.54 and 87.52 (CH), 85.10 and 85.03 (CH), 83.83 and 83.79 (CH), 71.02 (CH), 63.41 and 63.31 (CH₂), 61.59 (CH₂), 39.88 and 39.85 (CH₂), 36.41 and 36.37 (C), 25.43 (CH₃), 19.29 and 19.23 (CH₃). HRMS (ES⁺ TOF) for [MNa]⁺C₂₂H₃₀N₂O₆Na calculated: 441.20020 observed: 441.19960.

### 5-[1-(2-chlorophenyl)-2,2-(dimethyl)propoxymethyl]-2′-deoxyundine (MPB-1-49)

Heating 5-BrCH₂-dU (250 mg, 0.385 mmol) with α-tert-butyl-2-chlorobenzyl alcohol (3,3-dimethyl-1-(2-chloro)phenyl-1-propanol) (540 mg, 2.718 mmol) for 3 hours at 118° C. followed by treatment with TBAF (43 mg, 0.136 mmol) afforded after purification (method A) 19 mg (11%) of product as 1:1 mixture of diastereomers. ¹H NMR (400 MHz, CD₃OD) for diastereomers: δ 7.91 and 7.89 (2 s, 1H), 7.53 (d, 1H, J=7.6 Hz), 7.31 (m, 3H), 6.27 (m, 1H), 4.67 (s, 1H), 4.41 (m, 1H), 4.10 (m, 1H), 4.00 (m, 1H), 3.95 (m, 1H), 3.76 (m, 2H), 2.27 (m, 2H), 0.97 and 0.96 (2 s, 9H). ¹³C NMR (100 MHz, CD₃OD) for diastereomers: δ 163.50 and 163.47 (C), 150.68 (C), 139.10 and 138.68 (CH), 137.28 (C), 134.38 (C), 129.76 and 129.70 (CH), 128.89 and 128.80 (CH), 128.40 and 128.36 (CH), 111.36 and 111.10 (C), 109.90 (CH), 87.57 and 87.52 (CH), 85.24 and 85.07 (CH), 83.89 and 83.36 (CH), 70.97 (CH), 64.20 and 63.72 (CH₂), 61.54 (CH₂), 39.89 and 39.83 (CH₂), 36.37 and 36.33 (C), 25.15 (CH₃). HRMS (ESI) for [MNa]⁺C₂₁H₂₇³⁵ClN₂O₆Na calculated: 461.14499 observed: 461.14504.

### 5-[1-(2-bromomethyl)phenyl-2,2-(dimethyl)propoxymethyl]-2′-deoxyuridine (KB-1-135)

Heating 5-BrCH₂-dU (208 mg, 0.320 mmol) with α-tert-butyl-2-bromobenzyl alcohol (3,3-dimethyl-1-(2-bromo)phenyl-1-propanol) (389 mg, 1.607 mmol) for 1 hour at 112° C. Purification (method A) afforded 3 mg (2%) of product as 1:1 mixture of diastereomers. ¹H NMR (400 MHz, CD₃OD) for diastereomers: δ 7.89 and 7.86 (2 s, 1H), 7.54 (m, 2H), 7.36 (m, 1H), 7.18 (m, 1H), 6.25 (m, 1H), 4.64 and 4.63 (2 s, 1H), 4.40 (m, 1H), 4.04 (m, 2H), 3.92 (m, 1H), 3.75 (m, 2H), 2.24 (m, 2H), 0.97 and 0.96 (s, 9H). HRMS (ESI) for [MH]⁺C₂₁H₂₈⁷³BrN₂O₆ calculated: 483.11307 observed: 483.11264, C₂₁H₂₈⁸¹BrN₂O₆ calculated: 485.11103 observed: 483.11055; for [MNa]⁺C₂₁H₂₇⁷³BrN₂O₆Na calculated: 505.09502 observed: 505.09452, O₂₁H₂₇⁸¹BrN₂O₆Na calculated: 507.09297 observed: 507.09212.

### 5-[1-(2,6-dichlorophenyl)-2,2-(dimethyl)propoxymethyl]-2′-deoxyuridine (MPB-1-57)

Heating 5-BrCH₂-dU (158 mg, 0.243 mmol) with α-tert-butyl-2,6-dichlorobenzyl alcohol (3,3-dimethyl-1-(2,6-dichloro)phenyl-1-propanol) (113 mg, 0.486 mmol) for 3.5 hours at 102-104° C. followed by treatment with TBAF (77 mg, 0.244 mmol) afforded after purification (method A) 8 mg (7%) of product as 1:1 mixture of diastereomers. ¹H NMR (400 MHz, CD₃OD) for diastereomers (NOTE: due to the presence of two ortho-substituents, there is, apparently, restricted rotation of the 2,6-dichlorophenyl group around its 1-C-4-C axis, which thereby makes 3-H non-equivalent to 5-H): δ 7.93 and 7.87 (2 s, 1H), 7.43 (d, J=8.0 Hz, 1H), 7.41 (d, J=8.5 Hz, 1H), 7.26 (t, J=8.0 Hz, 1H), 6.30 (m, 1H), 5.08 and 5.06 (2 s, 1H), 4.40 (m, 1H), 4.10 (m, 2H), 3.94 (m, 1H), 3.72 (m, 2H), 2.30 (m, 1H), 2.18 (m, 1H), 1.06 (s, 9H). ¹³C NMR (100 MHz, CD₃OD) for diastereomers (NOTE: due to the presence of two ortho-substituents, there is, apparently, restricted rotation of the 2,6-dinitrophenyl group around its 1-C-4-C axis, which thereby makes 2-CNO₂ non-equivalent to 6-CNO₂, and accordingly, 3-CH non-equivalent to 5-CH) δ 163.51 and 163.44 (C), 150.72 (C), 139.16 and 139.11 (CH), 137.26 and 137.19 (C), 134.38 (C), 133.43 (C), 131.06 (CH), 128.96 and 128.93 (CH), 128.62 and 128.57 (CH), 110.94 (C), 87.53 and 87.43 (CH), 85.73 and 85.33 (CH), 85.12 and 84.93 (CH), 71.09 and 71.01 (CH), 63.75 and 63.62 (CH₂), 61.67 and 61.64 (CH₂), 39.84 and 39.74 (CH₂), 38.35 (C), 26.78 (CH₃). HRMS (ESI) [MNa]⁺C₂₁H₂₇³⁵Cl₂N₂O₆ calculated: 473.12462 observed: 473.12412; for [MNa]⁺C₂₁H₂₆³⁵Cl₂N₂O₆Na calculated: 495.10656 observed: 495.10611.

The di-tert-butylcarbinol-oxy-T analog was synthesized using mechanochemical conditions.³³

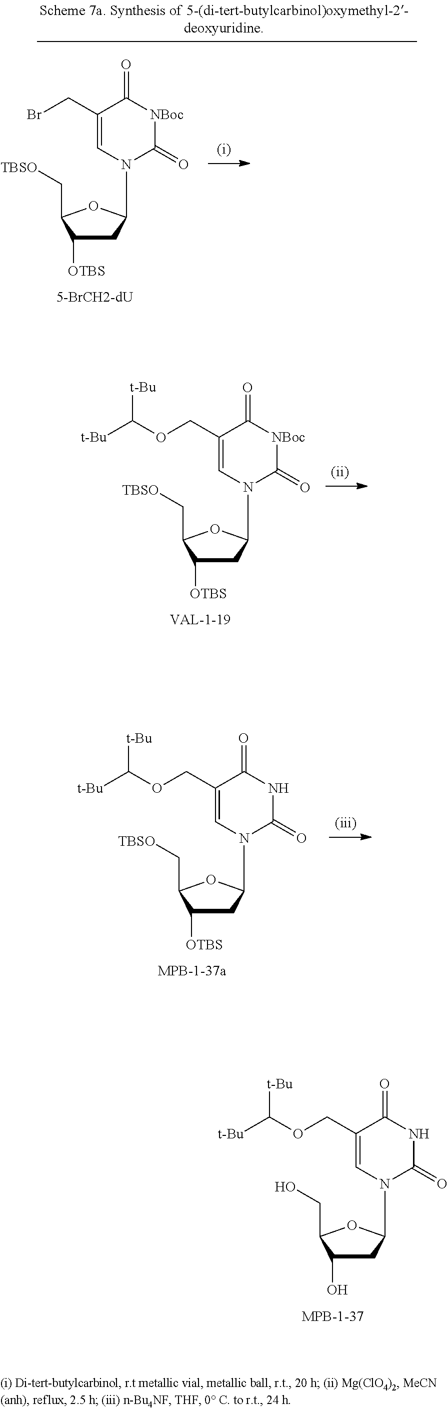

N³-tert-Butyloxycarbonyl-5-(di-tert-butylcarbinol)oxymethyl-3′,5′-bis-O-tert-butyldimethylsilyl-2′-deoxyuridine (VAL-1-19). 5-BrCH₂-dU (255 mg, 0.392 mmol) and di-tert-butylcarbinol (453 mg, 1.140 mmol) were placed in an iron screw-top vial equipped with a ball followed by vigorous shaking at room temperature for 20 hours under argon atmosphere. The contents of the vial were dissolved in ethyl acetate (1 mL) and mixed with silica (ca 500 mg). The solvent was evaporated, and the powder was applied onto a chromatography column (SiO₂, hexane/ethyl acetate=15:1 to 6:1) to afford 50 mg (18%) of crude product. ¹H NMR (500 MHz, CDCl₃) δ 7.64 (s, 1H), 6.27 (t, 1H, J=6.7 Hz), 4.49 (m, 1H), 4.44 (m, 2H), 3.95 (q, 2H, J=3.4 Hz), 3.92 (AB d, 1H, J=11.0 Hz), 3.75 (AB d, 1H, J=11.0 Hz), 2.82 (s, 1H), 2.33 (m, 2H), 1.62 (s, 9H), 1.05 (s, 18H), 0.91 (s, 18H), 0.10 (2 s, 6H), 0.09 (s, 6H). The product was not further characterized but introduced into the subsequent transformation as is.

### 5-(di-tert-butylcarbinol)oxymethyl-3′,5′-bis-O-tert-butyldimethylsilyl-2′-deoxyuridine (MPB-1-37A)

VAL-1-19 (50 mg, 0.070 mmol) was placed into a round bottom flask and purged with argon for 10 minutes. Anhydrous acetonitrile (10 mL) and magnesium perchlorate (2 mg, 0.009 mmol) were added, and the reaction mixture was stirred at reflux for 2.5 hours under argon atmosphere. The solvent was removed under reduced pressure; the crude product was dissolved in ethyl acetate (1 mL) and mixed with silica (ca 500 mg). The solvent was evaporated, and the powder was applied onto a chromatography column (SiO₂, hexane/ethyl acetate=8:1 to 4.1) to afford 22 mg (51%) of product. ¹H NMR (500 MHz, CDCl₃) δ 8.36 (s, 1H), 7.50 (s, 1H), 6.31 (dd, 1H, J=7.7, 5.9 Hz), 4.41 (s, 2H), 4.38 (m, 1H), 3.95 (m, 1H), 3.77 (AB dd, 1H, J=10.6, 4.7 Hz), 3.56 (AB dd, 1H, J=10.6, 7.0 Hz), 2.80 (s, 1H), 2.32 (m, 1H), 1.90 (m, 1H), 1.03 (s, 18H), 0.90 and 0.89 (2 s, 18H), 0.09 and 0.07 (2 s, 12H). ¹³C NMR (125 MHz, CDCl₃) δ 163.43 (C), 149.85 (C), 135.41 (CH), 107.73 (C), 96.13 (CH), 87.64 (CH), 85.31 (CH), 72.69 (CH), 68.70 (CH₂), 63.58 (CH₂), 40.04 (CH₂), 38.75 (C), 29.45 (CH₃), 29.20 (CH₃), 25.92 (CH₃), 17.98 (C), −4.70 (CH₃), −5.39 (CH₃).

### 5-(di-tert-butylcarbinol)oxymethyl-2′-deoxyuridine (MPB-1-37)

MPB-1-37A (22 mg, 0.036 mmol) was dissolved in tetrahydrofuran (2.5 mL) chilled at 0° C. by means of ice-water bath. Tetra-n-butylammonium fluoride trihydrate (28 mg, 0.090 mmol) was added, and the reaction mixture was stirred for 24 hours while gradually warming up to room temperature. The solvent was removed under reduced pressure; the crude product was dissolved in dichloromethane/methanol=10:1 (1 mL) and was mixed with silica (ca 200 mg). The solvent was evaporated, and the powder was applied onto a chromatography column (SO₂, dichloromethane/methanol=1:0 to 20:1) to afford 6 mg (43%) of product. ¹H NMR (500 MHz, CD₃OD) δ 7.93 (s, 1H), 6.32 (t, 1H, J=6.7 Hz), 4.39 (m, 3H), 3.93 (m, 1H), 3.72 (d, 2H, J=4.0 Hz), 2.84 (s, 1H), 2.31 (m, 1H), 2.19 (m, 1H), 1.06 (s, 18H). ¹³C NMR (125 MHz, CD₃OD) δ 163.49 (C), 150.77 (C), 137.59 (CH), 112.33 (C), 94.70 (CH), 87.49 (CH), 85.03 (CH), 71.13 (CH), 68.51 (CH₂), 61.77 (CH₂), 39.71 (CH₂), 38.28 (C), 28.35 (CH₃). HRMS (TOF ES⁺) for [M+Na]⁺C₁₉H₃₂N₂O₆Na⁺ calculated: 407.2158, observed: 407.2160.

Diversification at the 4-aromatic carbon was achieved by Sonogashira coupling of MPB-1-33 to a terminal alkyne, followed by other transformations, if applicable (Scheme 7b).

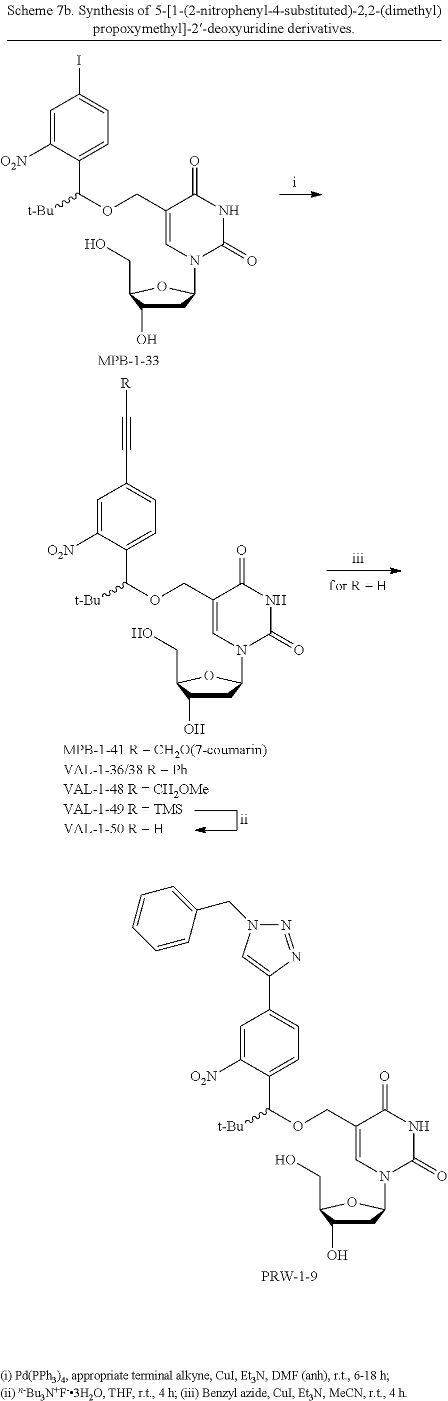

General procedure for Sonogashira coupling. 5-[1-(4-iodo-2-nitrophenyl)-2,2-(dimethyl)propoxymethyl]-2′-deoxyuridine (MPB-1-33) was dissolved in anhydrous N,N-dimethylformamide to make ca 0.4 M solution followed by addition of the appropriate alkyne (typically, 3.0 eq.), copper(I) iodide (0.2 eq.), diisopropylethylamine (17.0 eq.), and tetrakis(triphenylphosphine)palladium(C) (0.1 eq.) in the exact order as described. The reaction mixture was stirred for 6 hours under argon atmosphere at room temperature. The reaction mixture was then diluted by dichloromethane and methanol (each in the amount equal to the initial DMF volume) followed by addition of sodium hydrogen carbonate (17 eq.). After stirring for additional 2 hours, the volatiles were removed under reduced pressure and the residue was purified by column chromatography on silica gel using ethyl acetate/methanol system (typically, eluting from 1:0 to 20:1).

5-[1-(4-{3-(7-coumarin)oxy}proyn-1-yl-2-nitrophenyl)-2,2-(dimethyl)propoxymethyl]-2′-deoxyuridine (MPB-1-41). Treatment of 68 mg of MPB-1-33 with 7-(propargyl)oxycoumarin (48 mg, 0.239 mmol) followed by purification using dichloromethane/methanol=1:0 to 10:1 system yielded 33 mg (64%) of product as 1:1 mixture of diastereomers. ¹H NMR (400 MHz, CD₃CN) for diastereomers: δ 9.07 (br. s, 1H), 7.88 (s, 1H), 7.84 and 7.81 (2 d, J=9.5 Hz, 1H), 7.70 (m, 2H), 7.58 (d, J=8.4 Hz, 1H), 7.04 (m, 1H), 7.01 and 6.89 (2 d, J=2.6 Hz, 1H), 6.26 (d, J=9.5 Hz, 1H), 6.17 (m, 1H), 5.10 (s, 2H), 4.90 and 4.89 (2 s, 1H), 4.35 (m, 1H), 4.07 (m, 3H), 3.86 (m, 1H), 3.66 (m, 2H), 3.45 (br. s, 1H), 3.16 (br. s, 1H), 2.20 (m, 2H, overlapped with H₂O), 0.82 and 0.81 (2 s, 9H). ¹³C NMR (100 MHz, CD₃CN) for diastereomers: δ 162.34 and 162.32 (C), 160.66 and 160.51 (C), 155.69 (C), 150.56 (C), 150.34 (C), 150.23 (C), 143.69 (CH), 139.44 and 139.28 (CH), 134.82 and 134.79 (CH), 130.59 and 130.53 (CH), 129.41 (CH), 126.74 and 126.69 (CH), 122.09 (C), 115.80 (C), 115.62 and 115.51 (C), 113.39 and 113.36 (CH), 112.65 (CH), 110.68 and 110.52 (C), 101.99 (CH), 87.34 (CH), 85.77 (C), 85.10 and 84.99 (CH), 84.70 (C), 81.53 and 81.14 (CH), 70.89 and 70.83 (CH), 64.54 and 64.40 (CH₂), 61.59 (CH₂), 56.69 (CH₂), 39.80 (CH₂), 36.34 and 36.29 (C), 25.01 (CH₃). HRMS (ESI) for [MNa]⁺C₃₃H₃₃N₃O₁₁Na calculated: 670.20130, observed: 670.20310.

5-[1-(4-{2-phenylacetylenyl}-2-nitrophenyl)-2,2-(dimethyl)-1-propoxymethyl]-2′-deoxyuridine (VAL-1-36/38). Treatment of 38 mg (0.067 mmol) of MPB-1-33 with phenylacetylene (21 mg, 0.201 mmol) followed by purification using ethyl acetate/methanol=20:1 system yielded 12 mg (33%) of product as 1:1 mixture of diastereomers. ¹H NMR (400 MHz, CD₃OD) for diastereomers: δ 8.04 (d, 1H, J=7.7 Hz), 8.00 and 7.97 (2 s, 1H), 7.80 (m, 2H), 7.58 (m, 2H), 7.43 (m, 2H), 6.28 (m, 1H), 4.96 and 4.95 (2 s, 1H), 4.39 (m, 1H), 4.19 (m, 2H), 3.91 (m, 1H), 3.73 (m, 2H), 2.24 (m, 2H), 0.86 and 0.84 (2 s, 9H). ¹³C NMR (100 MHz, CD₃OD) for diastereomers: b 163.57 and 163.43 (C), 150.70 and 150.61 (C), 147.67 and 146.90 (C), 140.01 and 139.70 (CH), 137.74 (CH), 134.18 and 134.13 (CH), 131.36 (CH), 128.76 (CH), 128.26 (CH), 126.12 (CH), 126.02 (C), 123.64 (C), 122.23 (C), 110.86 and 110.60 (C), 91.16 (C), 87.59 (CH), 86.16 (C), 85.19 and 85.08 (CH), 81.66 and 81.04 (CH), 70.98 and 70.92 (CH), 64.60 and 64.32 (CH₂), 61.48 and 61.44 (CH₂), 39.92 and 39.85 (CH₂), 36.18 (C), 24.82 (CH₃). HRMS (ESI) for [MH]⁺C₂₉H₃₂N₃O₈ calculated: 550.21856, observed: 550.21839.

5-[1-(4-{3-methoxy}proyn-1yl-2-nitrophenyl)-2,2-(dim ethyl)-1-propoxymethyl]-2′-deoxyuridine (VAL-1-48). Treatment of 24 mg (0.042 mmol) of MPB-1-33 with methyl propardyl ether (15 mg, 0.209 mmol) followed by purification using dichloromethane/methanol=1:0 to 30:1 system yielded 10 mg (64%) of product as 1:1 mixture of diastereomers. ¹H NMR (400 MHz, CD₃OD) for diastereomers: δ 8.02 and 8.00 (2 s, 1H), 7.87 (m, 1H), 7.76 (AB d, J=8.2 Hz, 1H), 7.70 (m, 1H), 6.25 (m, 1H), 4.94 (s, 1H, note: overlapped with HDO), 4.40 (m, 1H), 4.35 (s, 2H), 4.17 (m, 2H), 3.92 (m, 1H), 3.75 (m, 2H), 3.44 (s, 3H), 2.25 (m, 2H), 0.84 and 0.82 (2 s, 9H). ¹³C NMR (100 MHz, CD₃OD) for diastereomers: δ 163.62 and 163.56 (C), 150.68 (C), 140.02 and 139.71 (CH), 137.15 and 138.83 (C), 134.37 and 134.33 (CH), 134.26 and 134.21 (C), 130.35 and 130.25 (CH), 126.22 and 126.22 (CH), 126.03 (CH), 110.81 and 110.56 (C), 87.60 and 87.34 (CH), 85.18 and 85.07 (CH), 83.21 (C), 81.61 and 81.03 (CH), 80.99 (C), 70.97 and 70.90 (CH), 64.58 and 64.30 (CH₂), 61.47 and 61.43 (CH₂), 59.42 (CH₂), 56.60 (CH₃), 39.92 and 39.84 (CH₂), 36.24 and 36.14 (C), 24.80 (CH₃). HRMS (ESI) for [MH]⁺C₂₅H₃₂N₃O₉ calculated: 518.21385, observed: 518.21342; [MNa]⁺C₂₅H₃₁N₃O₉Na calculated: 540.19580, observed: 540.19529.

5-[1-(4-{trimethylsilyl}acetylenyl-2-nitrophenyl)-2,2-(dimethyl)-1-propoxymethyl]-2′-deoxyuridine (VAL-1-49). Treatment of 87 mg (0.152 mmol) of MPB-1-33 with (trimethylsilyl)acetylene (60 mg, 0.607 mmol) followed by purification using ethyl acetate/methanol=1:0 to 100:1 system yielded 32 mg (38%) of product as 1:1 mixture of diastereomers. ¹H NMR (400 MHz, CD₃OD) for diastereomers: δ 8.01 and 8.00 (2 s, 1H), 7.84 (m, 1H), 7.74 (AB d, J=8.1 Hz, 1H), 7.68 (m, 1H), 6.26 (m, 1H), 4.93 and 4.92 (2 s, 1H), 4.40 (m, 1H), 4.18 (m, 2H), 3.92 (m, 1H), 3.74 (m, 2H), 2.24 (m, 2H), 0.83 and 0.82 (2 s, 9H), 0.26 (s, 9H). ¹³C NMR (100 MHz, CD₃OD) for diastereomers: δ 165.00 and 164.95 (C), 152.10 and 152.00 (C), 151.86 (C), 141.14 and 141.14 (CH), 135.86 and 135.75 (CH), 131.73 and 131.63 (CH), 129.91 (C), 127.84 and 127.74 (CH), 124.83 (C), 112.22 and 111.98 (C), 103.33 (C), 97.79 (C), 89.01 (CH), 86.61 and 86.51 (CH), 83.02 and 82.42 (CH), 72.38 and 72.31 (CH), 66.00 and 65.70 (CH₂), 62.88 and 62.84 (CH₂), 41.33 and 41.25 (CH₂), 37.66 and 37.56 (C), 26.20 (CH₃), −0.25 (CH₃). HRMS (ESI) for [MH]⁺C₂₆H₃₆N₃O₈Si calculated: 546.22717, observed: 546.22670; [MNa]⁺C₂₆H₃₅N₃O₈SiNa calculated: 568.20911, observed: 568.20911.

Procedure for TMS removal. 5-[1-(4-{trimethylsilyl}acetylenyl-2-nitrophenyl)-2,2-(dimethyl)propoxymethyl]-2′-deoxyuridine (VAL-1-49, 30 mg, 0.055 mmol) was dissolved in tetrahydrofuran (2.5 mL) followed by addition of tetra-n-butylammonium fluoride trihydrate (26 mg, 0.083 mmol). The reaction mixture was stirred for 6 hours, then concentrated under reduced pressure, and the residue was purified by column chromatography on silica gel using ethyl acetate/methanol=1:0 to 40:1 to afford 10 mg (38%) of 5-[1-(4-acetylenyl-2-nitrophenyl)-2,2-(dimethyl)propoxymethyl]-2′-deoxyuridine (VAL-1-50) as 1:1 mixture of diastereomers. ¹H NMR (400 MHz, CD₃OD) for diastereomers: δ 8.02 and 8.00 (2 s, 1H), 7.90 (m, 1H), 7.76 (AB d, J=8.2 Hz, 1H), 7.65 (m, 1H), 6.26 (m, 1H), 4.94 and 4.93 (2 s, 1H), 4.40 (m, 1H), 4.18 (m, 2H), 3.92 (m, 1H), 3.74 (m, 3H), 2.24 (m, 2H), 0.84 and 0.82 (2 s, 9H). ¹³C NMR (100 MHz, CD₃OD) for diastereomers: δ 163.60 and 163.55 (C), 150.69 and 150.61 (C), 150.48 (C), 140.01 and 139.91 (CH), 134.73 and 134.52 (CH), 130.94 and 130.26 (CH), 128.46 (C), 126.71 and 126.61 (CH), 122.79 (C), 110.82 and 110.57 (C), 87.61 (CH), 85.20 and 85.10 (CH), 81.61 and 81.01 (CH), 80.48 (C), 80.17 (CH), 70.97 and 70.90 (CH), 64.57 and 64.30 (CH₂), 61.47 and 61.43 (CH₂), 39.93 and 39.86 (CH₂), 36.93 and 36.14 (C), 24.78 (CH₃). HRMS (ESI) for [MH]⁺C₂₃H₂₈N₃O₈ calculated: 474.18764, observed: 474.18715; [MNa]⁺C₂₃H₂₇N₃O₈Na calculated: 496.16958, observed: 496.16915.

Procedure for click reaction. 5-[1-(4-acetylenyl-2-nitrophenyl)-2,2-(dimethyl)propoxymethyl]-2′-deoxyuridine (VAL-1-50, 3.8 mg, 0.008 mmol) was dissolved in acetonitrile (2 mL) followed by addition of benzyl azide (2 mg, 0.014 mmol), diisopropylethylamine (10 mg, 0.08 mmol) and copper(I) iodide (0.1 mg, 0.0008 mmol). The reaction mixture was stirred for 2 hours under argon atmosphere at room temperature. The reaction mixture was then concentrated under reduced pressure and the residue was purified by column chromatography on silica gel using dichloromethane/methanol=1:0 to 30:1 to afford 3.7 mg (76%) of product PRW-1-9 as 1:1 mixture of diastereomers. ¹H NMR (400 MHz, CD₃OD) for diastereomers: δ 8.51 (s, 1H), 8.27 (m, 1H), 8.07 (s, 1H), 8.00 (d, 1H, J=8.3 Hz), 7.83 (d, 1H, J=8.3 Hz), 7.58 (m, 2H), 7.43 (m, 2H), 6.25 (m, 1H), 5.67 (s, 1H), 4.97 and 4.96 (2 s, 1H), 4.42 (m, 1H), 4.20 (m, 2H), 3.94 (m, 1H), 3.76 (m, 2H), 2.26 (m, 2H), 0.87 and 0.86 (2 s, 9H). HRMS (ESI) for [MH]⁺C₃₀H₃₅N₆O₈ calculated: 550.24747, observed: 607.25119.

5-Aminoxy thymidine nucleoside analogs (Formula 1 b) will be synthesized similarly, by treatment of 5-BrCH₂-dU with an appropriate amine or an ionized thiol, followed by removal of N³-Boc and TBS groups (Scheme 8).

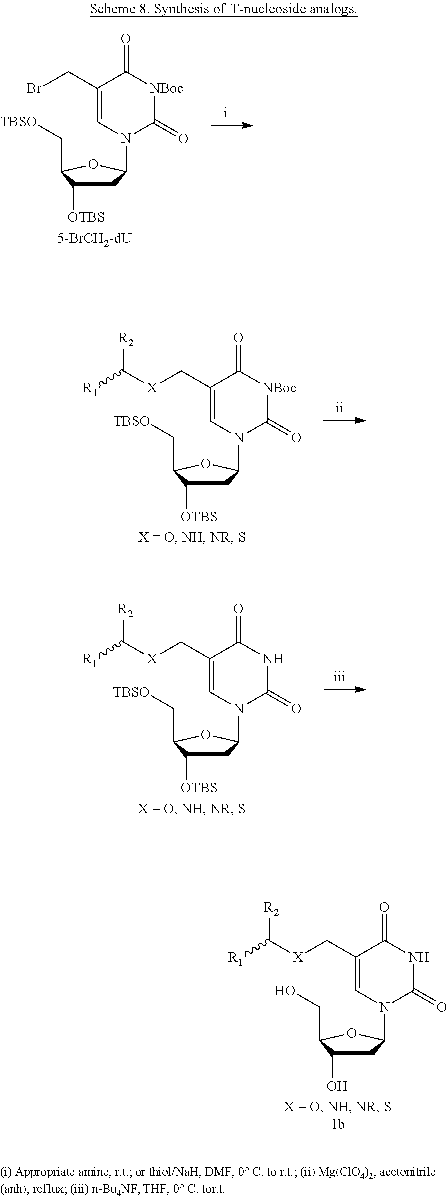

Synthesis of C-Nucleoside Analogs

Several examples of 2′-deoxycytidine nucleoside analogs (Formula 2b) have been synthesized similarly to the previously described procedure from bis-TBSO-protected T-nucleosides by conversion of 2′-deoxyuridines into 2′-deoxycytidines (Scheme 9).

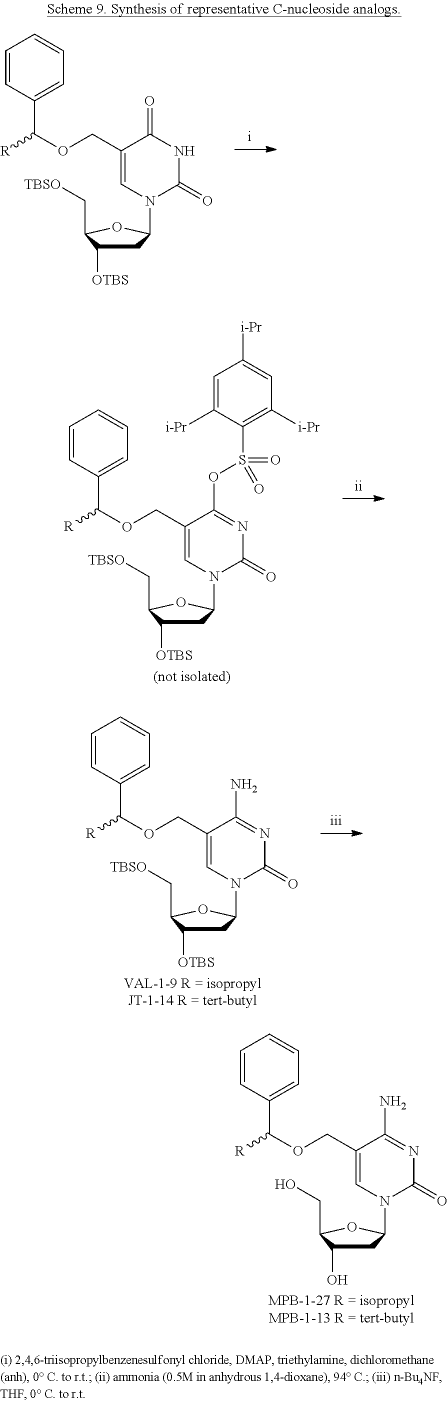

General procedure for conversion of 2′-deoxyuridines into 2′-deoxycytidines.

Bis-TBSO-protected T-nucleosides^{16,17} were dissolved in anhydrous dichloromethane (ca 0.02 M) followed by addition of 4-dimethylaminopyridine (DMAP) (1.03 eq.) and triethylamine (9.00 eq.). The mixture was cooled down to 0° C. by means of ice-water bath and triisopropylbenzenesulfonylchloride (9 eq.) was added. The reaction mixture was stirred under argon atmosphere for 18 hours while gradually warming up to room temperature. The solvent and the excess of triethylamine was removed under reduced pressure, and 0.5 M solution of ammonia in dioxane (36 eq.) was added to the residue. The reaction mixture was transferred to a high pressure tube, sealed, and heated at 94° C. for 17 hours. After cooling down the solvent was removed under reduced pressure and the residue was purified by silica gel to yield the product as a waxy solid.

5-[1-(phenyl)-2-(methyl)propoxymethyl]-3′,5′-bis-(tert-butyl)dimethylsilyl-2′-deoxycytidine (VAL-1-9 and MPB-1-19). Treatment of 5-[1-(phenyl)-2-(methyl)propoxymethyl]-3′,5′-bis-(tert-butyl)dimethylsilyl-2′-deoxyuridine (120 mg, 0.195 mmol) with DMAP (23 mg, 0.201 mmol), triethylamine (178 mg, 1.758 mmol), and triisopropylbenzenesulfonyl chloride (532 mg, 1.758 mmol) in anhydrous CH₂Cl₂ (8.5 mL) followed by treatment with 0.5 M ammonia in dioxane (14.06 mL, 7.030 mmol) afforded after purification 84 mg (69%) of product as 1:1 mixture of diastereomers. ¹H NMR (400 MHz, CDCl₃) for diastereomers: δ 7.51 and 7.49 (2 s, 1H), 7.34 (m, 3H), 7.24 (m, 2H), 6.27 (m, 1H), 4.30 (m, 1H), 4.07 (m, 2H), 3.90 (m, 2H), 3.92 (m, 1H), 3.74 (m, 2H), 2.41 (m, 1H), 1.93 (m, 1H), 1.02 (m, 3H, CH₃), 0.88 and 0.87 (2 s, 9H, 3 CH₃), 0.82 and 0.79 (2 s, 9H, 3 CH₃), 0.70 (m, 3H, CH₃), 0.07 and 0.06 (2 s, 3H, CH₃), 0.05 and 0.04 (2 s, 3H, CH₃), 0.01 and −0.02 (2 s, 3H, CH₃), −0.04 and −0.07 (2 s, 3H, CH₃). ¹³C NMR (100 MHz, CDCl₃) for diastereomers: δ 163.52 (C), 155.16 (C), 139.88 and 139.80 (CH), 139.04 and 138.92 (C), 128.57 and 128.51 (CH), 128.12 (CH), 127.62 and 127.46 (CH), 102.28 (C), 88.11 and 87.92 (CH), 87.76 and 87.72 (CH), 86.31 and 86.10 (CH), 72.14 and 71.75 (CH), 65.76 and 65.32 (CH₂), 62.89 and 62.73 (CH₂), 42.16 and 42.06 (CH₂), 34.47 (CH), 19.40 (CH₃), 18.97 (CH₃) 18.31 (C), 18.0 (C), −4.58 (CH₃), −4.87 (CH₃), −5.44 (CH₃), −5.48 (CH₃).

5-[1-(phenyl)-2,2-(dimethyl)propoxymethyl]-3′,5′-bis-(tert-butyl)dimethylsilyl-Z-deoxycytidine (JT-1-14). Treatment of 5-[1-(phenyl)-2,2-(dimethyl)propoxymethyl]-3′,5′-bis-(tert-butyl)dimethylsilyl-Z-deoxyuridine¹⁷ (95 mg, 0.150 mmol) with DMAP (17 mg, 0.154 mmol), triethylamine (136 mg, 1.348 mmol), and triisopropylbenzenesulfonyl chloride (409 mg, 1.348 mmol) in anhydrous CH₂Cl₂ (7.0 mL) followed by treatment with 0.5 M ammonia in dioxane (10.80 mL, 5.400 mmol) afforded after purification 24 mg (69%) of product as 1:1 mixture of diastereomers. ¹H NMR (400 MHz, CDCl₃) for diastereomers: δ 7.43 and 7.42 (2 s, 1H), 7.34 (m, 2H), 7.24 (m, 3H), 6.26 (m, 1H), 5.99 (br. s, 2H), 4.30 (m, 1H), 4.17 (m, 1H), 3.96 (m, 3H), 3.71 (m, 2H), 2.44 (m, 1H), 1.90 (m, 1H), 0.92 and 0.90 (2 s, 9H), 0.89 (s, 9H), 0.82 and 0.80 (2 s, 9H), 0.08 and 0.07 (2 s, 3H), 0.07 and 0.06 (2 s, 3H), 0.02 and 0.00 (2 s, 3H), −0.04 and −0.06 (2 s, 3H). ¹³C NMR (100 MHz, CD₃OD) for diastereomers: 165.15 (C), 155.15 (C), 139.90 and 139.65 (CH), 138.24 and 138.22 (C), 128.48 and 128.42 (CH), 127.96 and 127.91 (CH), 127.85 and 127.78 (CH), 102.61 and 102.13 (C), 90.20 and 89.16 (CH), 87.89 and 87.63 (CH), 86.36 and 86.10 (CH), 72.23 and 71.81 (CH), 66.29 and 65.72 (CH₂), 62.90 and 62.76 (CH₂), 41.12 and 41.96 (CH₂), 34.56 and 35.42 (C), 26.42 and 25.72 (CH₃), 25.83 and 25.75 (CH₃), 18.25 and 18.29 (C), 17.99 (C), −4.59 (CH₃), −4.88 (CH₃), −5.39 and −5.43 (CH₃), −5.46 and −5.51 (CH₃).

General Procedure for TBS Group Removal.

Bis-TBSO-protected O-nucleosides were dissolved in tetrahydrofuran (ca 0.02 M) followed by addition of tetra-n-butylammonium fluoride trihydrate (7 eq.) at 0° C. The reaction mixture was stirred for 18 hours while gradually warming up to room temperature. The solvent was removed under reduced pressure and the residue was purified by silica gel and then by C8 reverse-phase column chromatography to yield the product as a waxy solid.

5-[1-(phenyl)-2-(methyl)propoxymethyl]-2′-deoxycytidine (MPB-1-12 and MPB-1-27). Treatment of VAL-1-9 (84 mg, 0.135 mmol) with tetra-n-butylammonium fluoride trihydrate (319 mg, 1.011 mmol) afforded after purification 27 mg (51%) of product as 1:1 mixture of diastereomers. ¹H NMR (400 MHz, CD₃OD) for diastereomers: δ 7.86 and 7.84 (2 s, 1H), 7.35 (m, 5H), 6.23 (m, 1H), 4.36 (m, 1H), 4.16 (m, 2H), 3.95 (m, 2H), 3.74 (m, 2H), 2.36 (m, 1H), 2.10 (m, 1H), 1.93 (m, 1H), 1.04 (2 d, 3H, J=6.7 Hz), 0.72 (d, 3H, J=6.8 Hz). ¹³C NMR (100 MHz, CD₃OD) δ 165.01 (C), 156.53 (C), 140.59 and 140.51 (CH), 140.37 (C), 128.05 and 127.98 (CH), 127.49 and 127.42 (CH), 127.37 and 127.32 (CH), 103.80 (C), 87.50 and 87.46 (CH), 87.10 and 86.64 (CH), 86.14 and 86.03 (CH), 70.55 and 70.47 (CH), 64.61 (CH₂) and 64.23 (CH₂), 61.30 and 61.26 (CH₂), 40.77 and 40.67 (CH₂), 34.51 and 34.44 (CH), 18.18 (CH₃), 18.01 and 17.99 (CH₃). HRMS (ESI) for [MH]⁺ calculated: 390.20235, observed: 390.20247.

5-[1-(phenyl)-2,2-(dimethyl)propoxymethyl]-2′-deoxycytidine (VAL-1-13, also JT-1-18). Treatment of JT-1-14 (18 mg, 0.195 mmol) with tetra-n-butylammonium fluoride trihydrate (319 mg, 1.011 mmol) afforded after purification 8 mg (68%) of product as 1:1 mixture of diastereomers. ¹H NMR (400 MHz, CD₃OD) for diastereomers: δ 7.82 and 7.78 (2 s, 1H), 7.34 (m, 5H), 6.22 (m, 1H), 4.35 (m, 1H), 4.23 (m, 2H), 4.04 (m, 1H), 3.93 (m, 1H), 3.72 (m, 2H), 2.36 (m, 1H), 2.09 (m, 1H), 0.92 and 0.91 (2 s, 9H). HRMS (ESI) for [MH]⁺ calculated: 404.21800, observed: 404.21811.

Additional 2′-deoxycytidine nucleoside analogs (Formula 2b) will be synthesized similarly from thymidine analogs 1 b (Scheme 10).

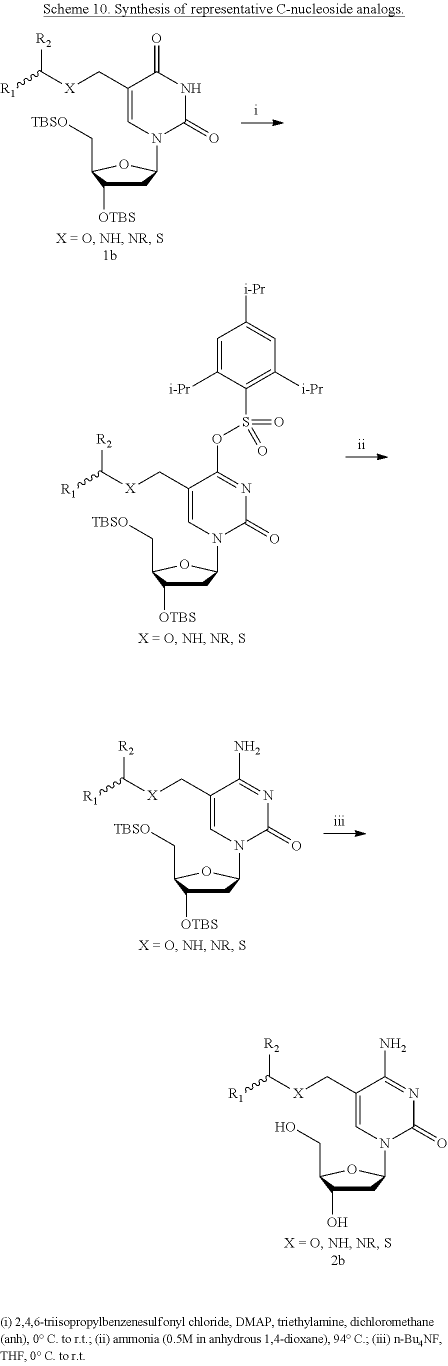

N-Alkylated adenine nucleoside analogs (Formula 3b) will be synthesized as described previously.¹⁴ Thus, commercially available inosine will undergo a reaction with an appropriate amine mediated by (benzotriazol-1-yloxy)tris(dimethylamino)phosphonium hexafluorophosphate (BOP), as reported previously (Scheme 11).

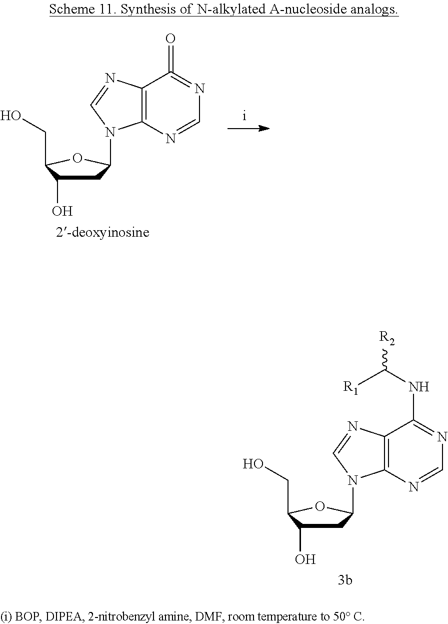

The synthesis of 7-deaza-7-substituted adenine nucleoside analogs (Formula 4a) will follow the already established routine for nucleosides. Briefly, 6-chloro-7-iodo-7-deazapurine will undergo coupling to 1′-chloro-3′,5′-bis-toluoyl-2′deoxyribose to produce a nucleoside. Further synthesis will include same transformations as that of the corresponding nucleobases (Scheme 5), plus TBS protection-deprotection of 3′ and 5′-hydroxyls of the sugar moiety (Scheme 12).

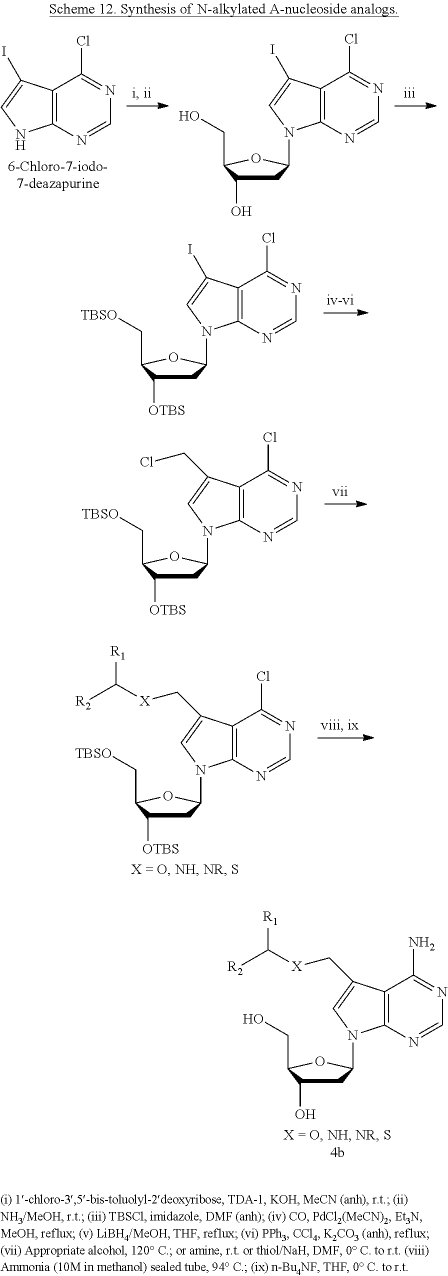

Likewise, the synthesis of 7-deaza-7-substituted-2′-deoxyguanosine nucleoside analogs (formula 5b) will be similar to that of the corresponding nucleobases, with the inclusion of the sugar coupling and TBS protection of 3′ and 5′-hydroxyls, which is an already established protocol (Scheme 13).

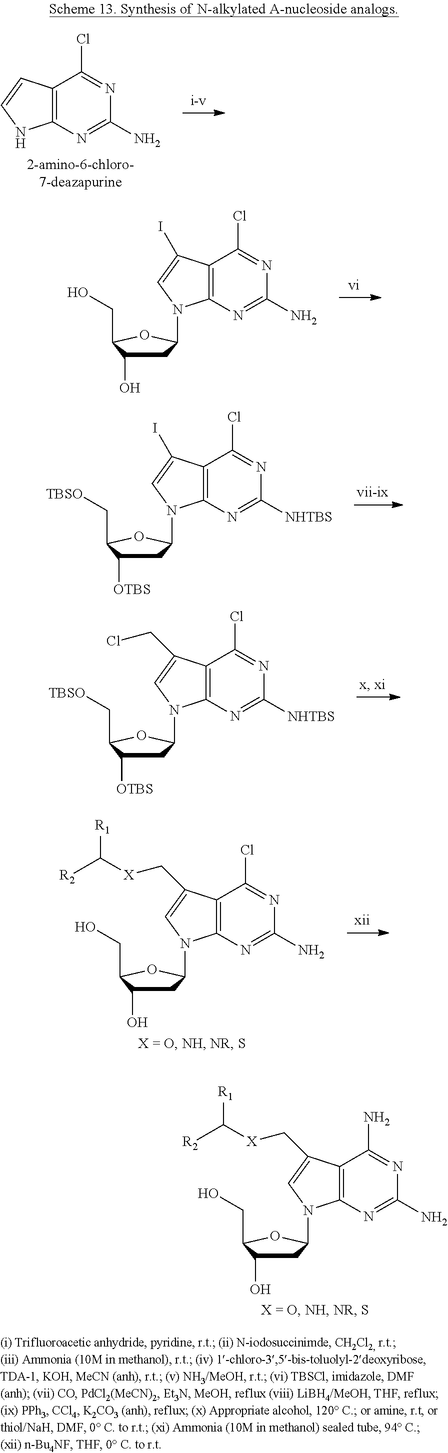

Design and Synthesis of Monomeric β-Cyclodextrin-Nucleoside Conjugates.

The most potent anti-cancer nucleosides from above will be conjugated to a derivative of benzaldehyde diacetal derivative bearing a water-soluble oligomeric chain (e.g. polyethylene glycol, PEG) via an acid-labile linker known to break at pH of tumor tissues, to form a conjugate, followed by attachment to a 6^{A},6^{D}-diazido-β-cyclodextrin using click-chemistry, which has been successfully applied to achieve functionalization of this system, and screening in vitro. Analogous monomers with a targeting ligand attached to β-cyclodextrin will be synthesized by click-reaction with another azido group and screened against the specific cancer cell lines to examine the effect on cellular membrane permeability. Scheme 14a and 14b illustrate the formation of exemplary conjugates.

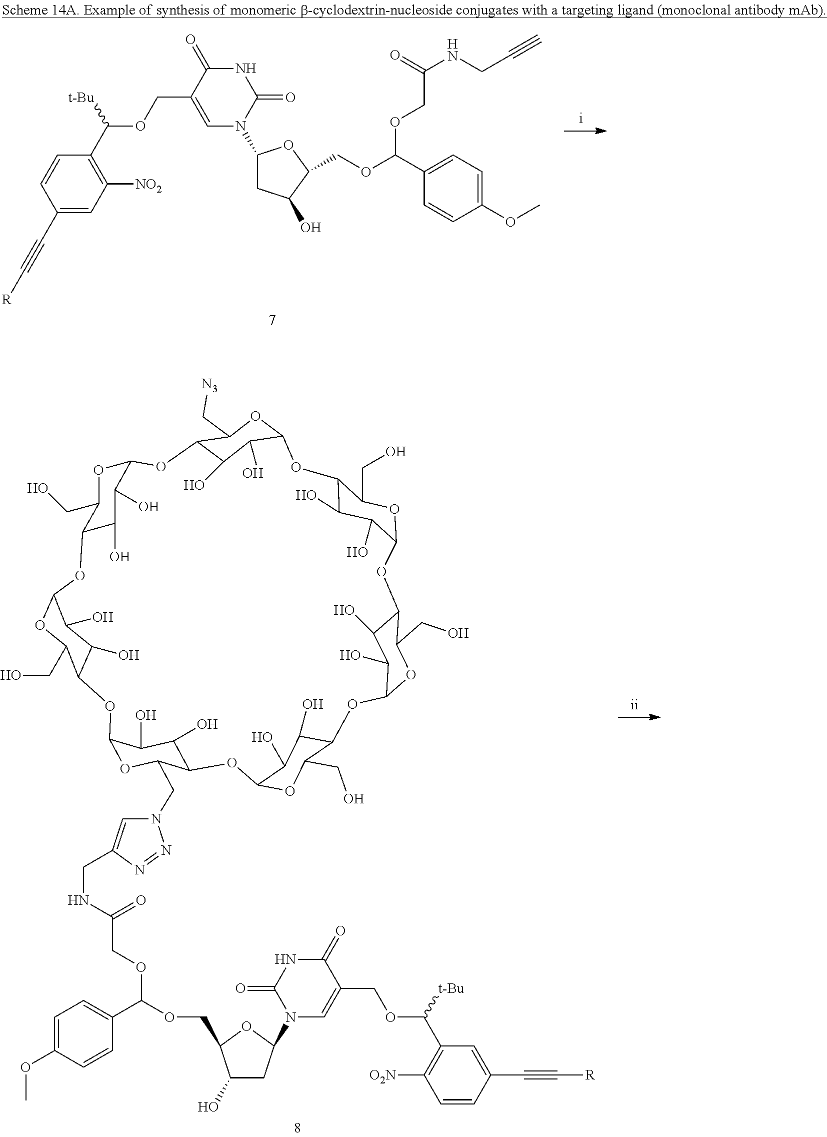

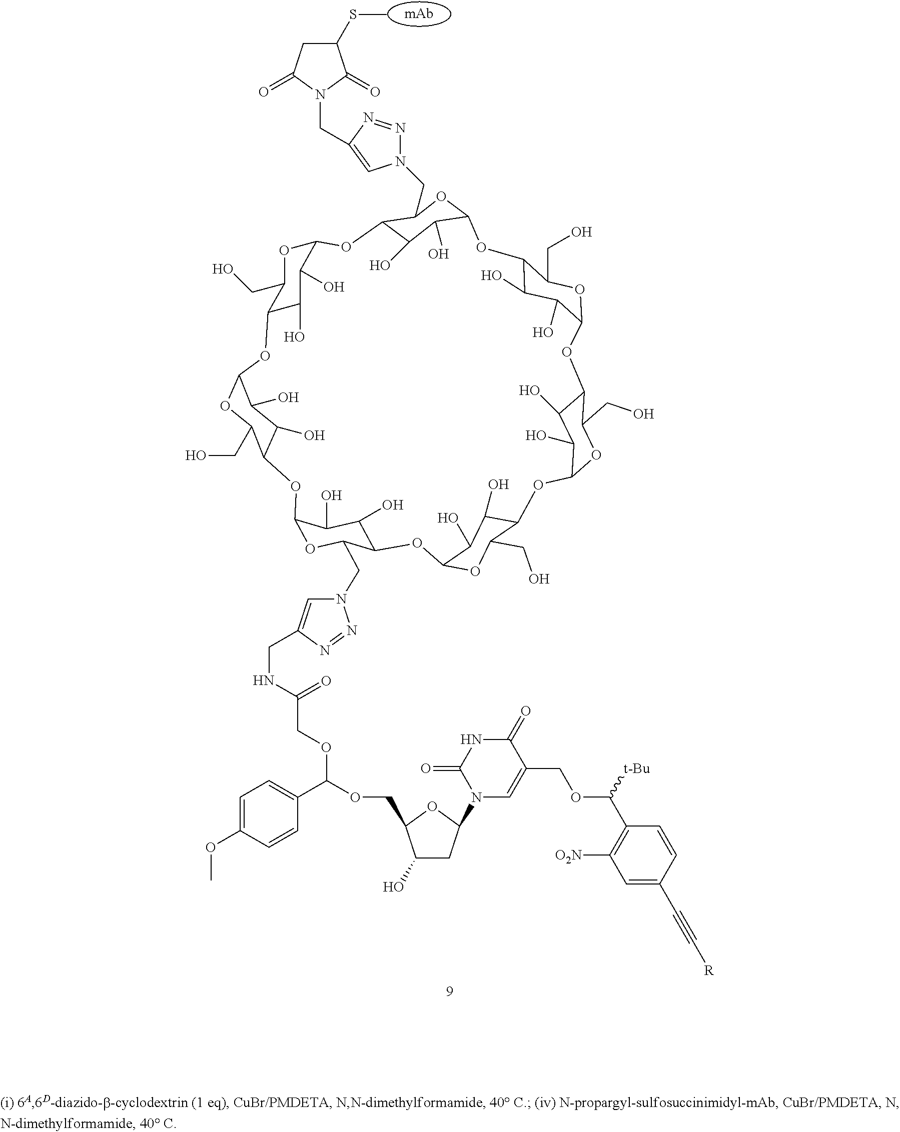

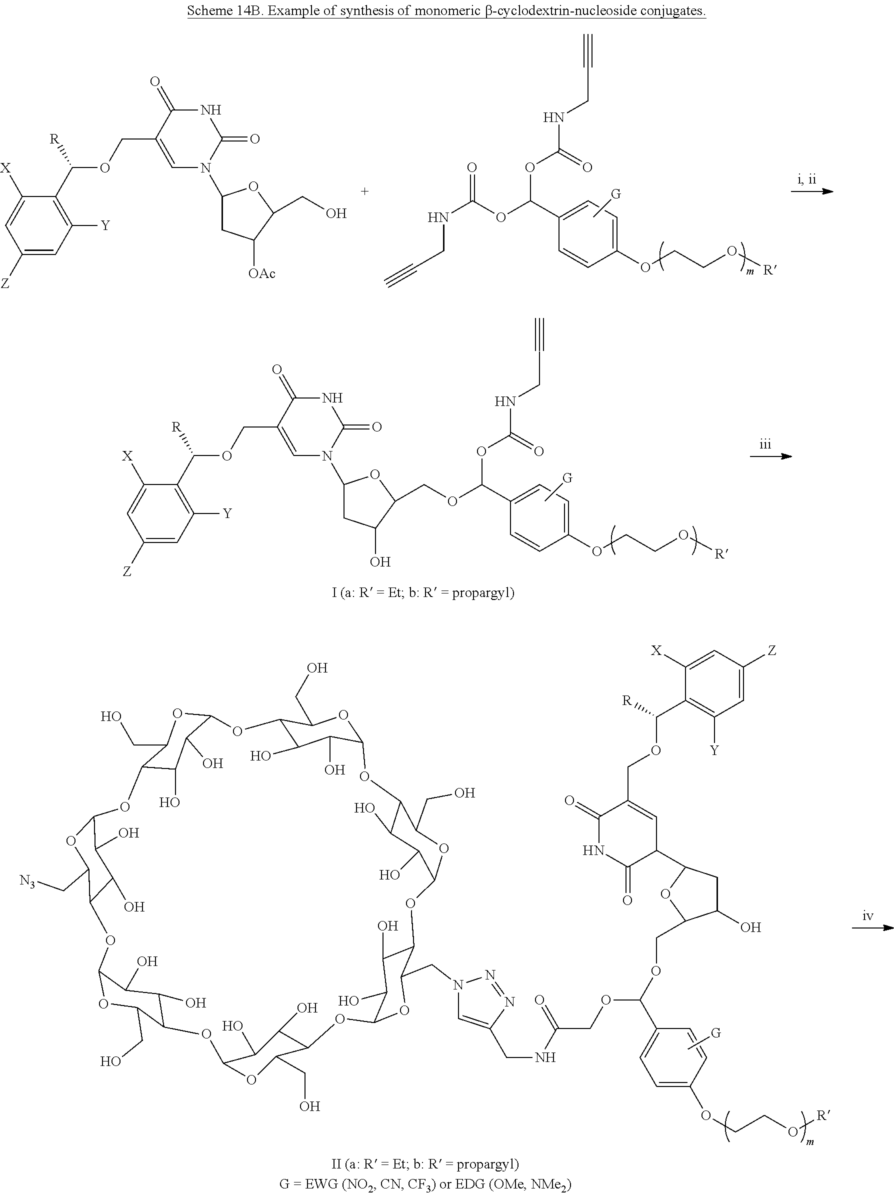

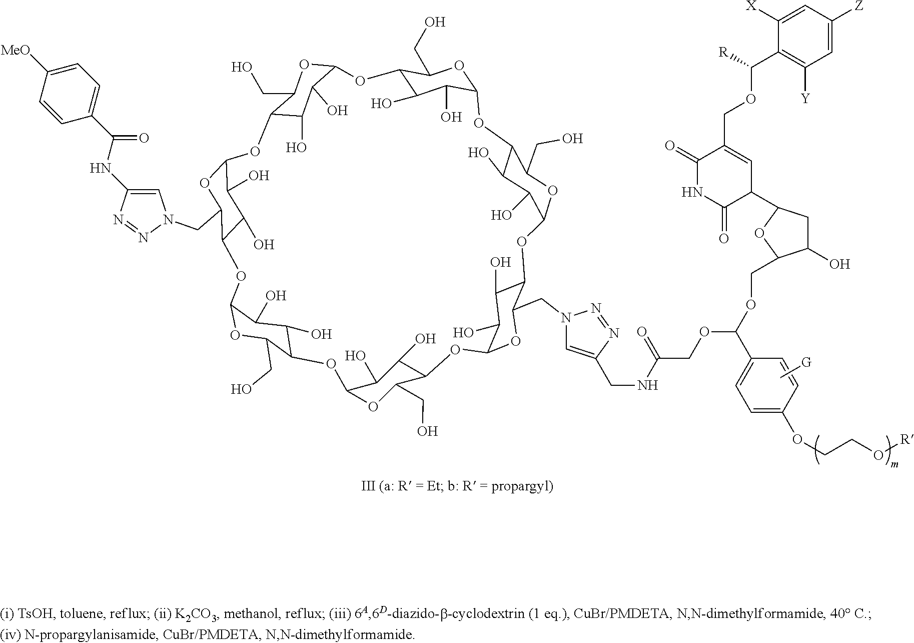

Synthesis of β-cyclodextrin-nucleoside-targeting ligand conjugated water-soluble polymers capable of self-assembly into nanoparticles. The nucleoside-amphiphilic oligomer conjugates (Ib in Scheme 14B) will be co-polymerized with 6^{A},6^{D}-diazido-β-cyclodextrin using click reaction (Scheme 15). 6^{A}-Azido-6^{D}-target ligand-β-cyclodextrin adducts will serve as chain terminators. Formation of an inter-strand inclusion complex between the hydrophobic cavity of β-CD and the 5(7)-benzyloxy terminating moiety of the attached nucleosides is expected to be the driving force for self-assembly of the resulting polymers into nanoparticles (Scheme 15) of the appropriate size to avoid renal clearance. The resulting drug candidates may be screened in vivo using tumor-bearing laboratory animals to determine their

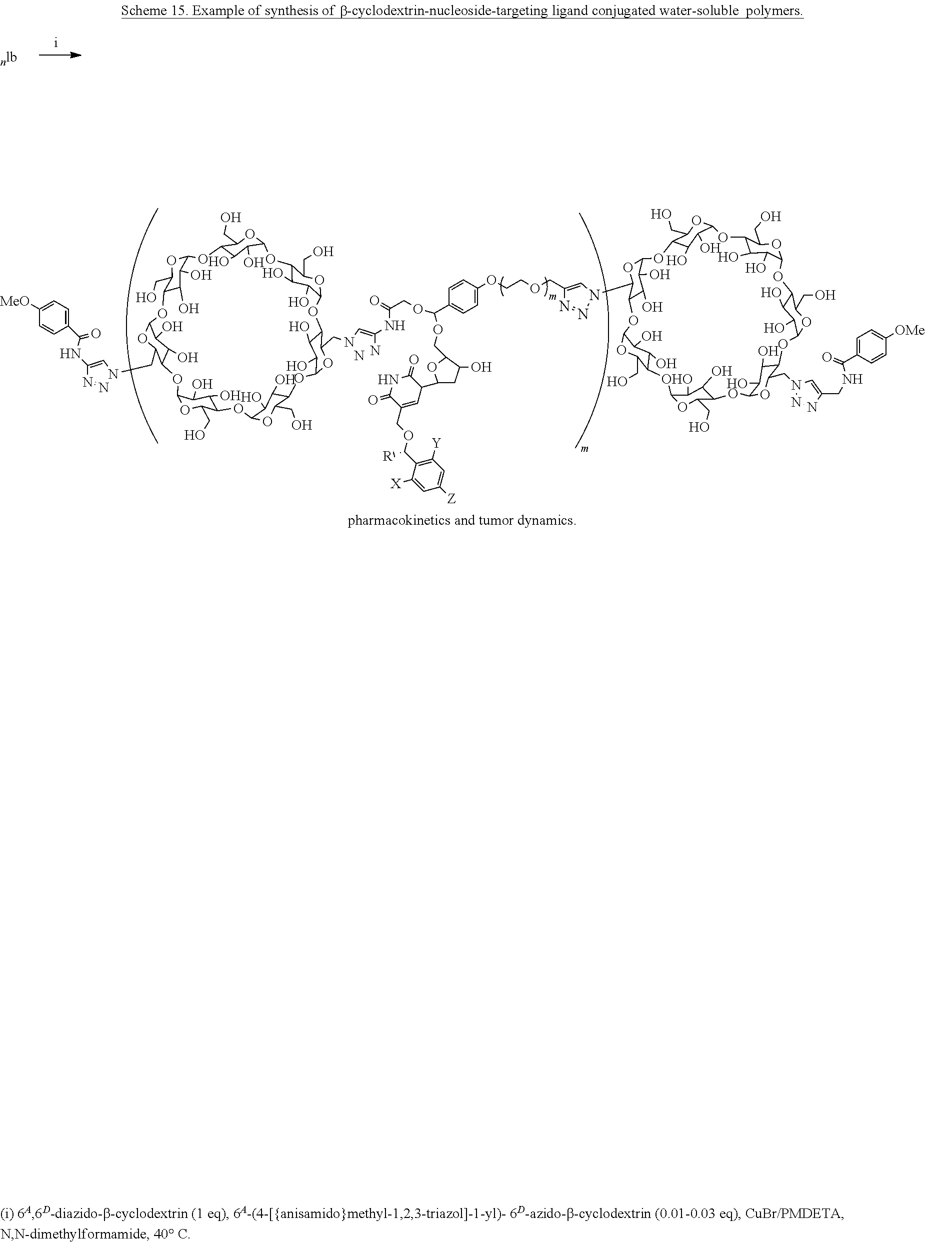

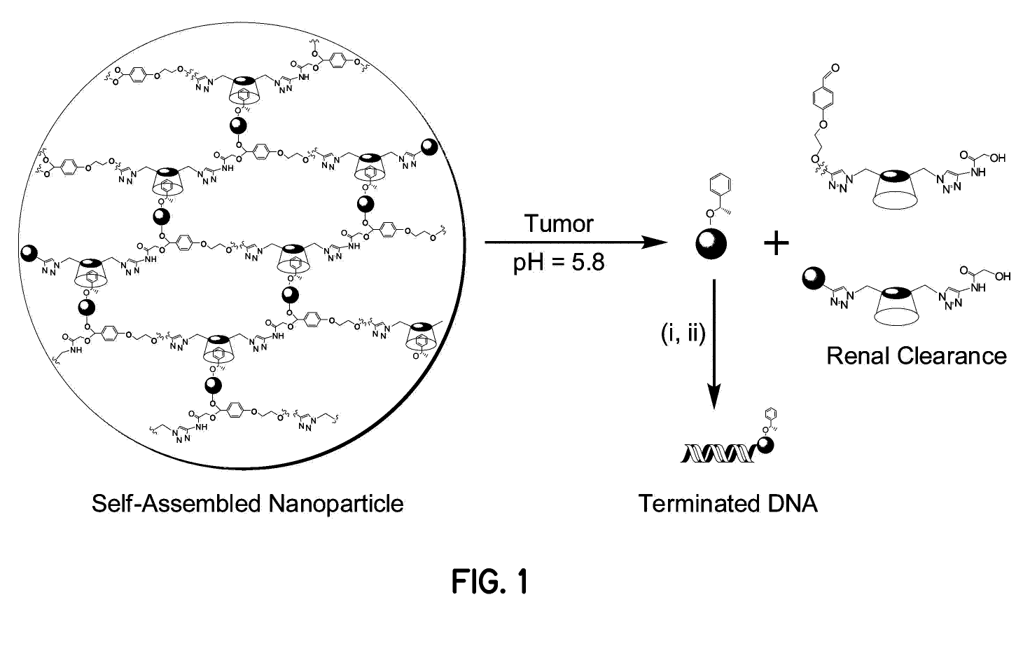

FIG. 1 illustrates the proposed therapeutic function of the novel anti-cancer agents. In the acidic pH of the tumor, the acid labile linker will release the nucleobase which results in the dissociation of the nanoparticle. The modified nucleobase undergoes (i) enzymatic 5′-triphosphorylation and (ii) incorporation into growing DNA strand, which terminates the elongation of the DNA strand. The remainder of the dissociated nanoparticle may be removed by normal renal clearance pathways.

The drug loading can be increased by attachment of additional chemotherapeutic agents to other OH groups of the β-cyclodextrin moiety via an acid-labile linker.

### Example 1

### Biological Studies

The cyctoxicity of compounds was determined in MCF 7 (breast cancer) cells.

MCF7 cells were grown in RPMI 1640 media with 10 nM estrogen and 1 mM insulin. Cells were tyripsinized and resuspended at a density of 2.2×10⁴ cells per mL. 500 μL of this suspension was added to each well in a 24 well plate. The plates were incubated at 37° C. and 5% CO₂ atmosphere overnight. The media was changed, and plates were dosed in triplicate with compound dissolved in DMSO. Cells were dosed not to exceed 0.5% DMSO in solution. Cells were dosed to a final concentration of 100, 50, 25, 12.5, and 6.25 μM of compound. 5-Flurouracil was used as a positive control and dosed in the same manner. Plates were incubated for 65 hours prior to the addition of MTT solution. 500 μL of a 193 μg/mL MTT and media solution was added to each well. Plates were incubated for 3 hours. The MTT solution was removed and 500 μL of DMSO was added to each well. Cells were imaged using GS 800 Bio Rad scanner and Quality One Software. IC₅₀ curves were determined by plotting viability verses compound concentration. Kaleidagraph software was used to calculate the R value for each logarithmic curve fitting.

Results

The first generation T-derivatives whose syntheses have been described in Schemes 7 and 7a have exhibited the following cytotoxicity in the breast cancer MCF7 cells, characterized by their IC₅₀ values (Table 1). The preliminary studies have revealed that important features of the pharmacophore are: (1) a bulky group in the closest proximity to the benzylic α-carbon; (2) the presence of an electron withdrawing substituent at the ortho-position of the phenyl ring, such as nitro group. This, the first generation lead compound was VAL-1-15/45, with R₁=t-Bu and R₂=2-nitrophenyl, as it has exhibited the lowest IC₅₀ value.

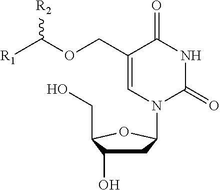

TABLE 1
Effect of base-modified T-nucleosides on viability of MCF7 cells from the MTT assay: compound (IC₅₀ in μM)

| | R₁ | | | | | | |
|---|---|---|---|---|---|---|---|
| R₂ | Me | i-Pr | t-Bu | Ph | neo-Am | Cy | n-Hex |
| 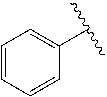 [SMILES: *c1ccccc1] | VAL-1-10 (>200) | MPB-1-25 (>150) | JT-1-10 (188 ± 3) | KB-1-10 (160 ± 1) | VAL-1-16 (123 ± 9) | PRW-1-13 (93 ± 12) | VAL-1-16 (50 ± 7) |
| 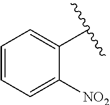 [SMILES: *c1ccccc1[N+](=O)[O-]] | VAL-1-25 (~170) | VAL-1-26 (~150) | VAL-1-15/27/42 (42 ± 6) | KB-1-85 (50 ± 5) | KB-1-91 (62 ± 6) | PRW-1-18 (63 ± 6) | KB-1-91 (72 ± 1) |

It is noteworthy that the presence of at least one aryl group appeared essential for activity. Derivatives with R₁=R₂=i-Pr or Bu were ineffective (Scheme 37). Indeed, the IC₅₀ values of the non-aryl-derivatized T-analogs shown below in breast cancer MCF7 cells were both greater than 120 μM.

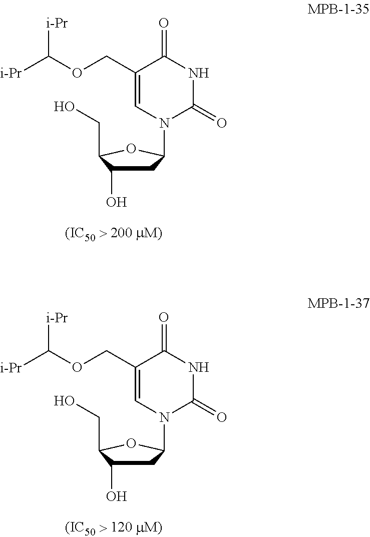

Further, the effect of varying the ortho-substituent in the aryl ring or add the same substituent in the other ortho-position was also investigated. The IC₅₀ values (Table 2) revealed that while both the bulkiness and the increased electron withdrawing character of the substituent improves the cytotoxicity, introduction of another ortho-substituent does not always have positive effect.

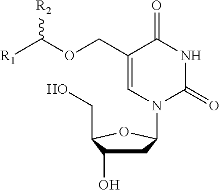

TABLE 2
Effect of ortho-substitution in T-nucleoside derivatives on viability of MCF7 cells from the MTT assy: compound (IC₅₀ in μM)

| | | R₁ | | | | | |
|---|---|---|---|---|---|---|---|
| R₂ | 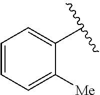 [SMILES: *c1ccccc1C] | 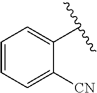 [SMILES: *c1ccccc1C#N] | 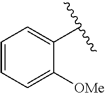 [SMILES: *c1ccccc1OC] | 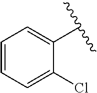 [SMILES: *c1ccccc1Cl] | 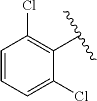 [SMILES: *c1c(Cl)cccc1Cl] | 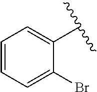 [SMILES: *c1ccccc1Br] | 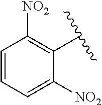 [SMILES: *c1c([N+](=O)[O-])cccc1[N+](=O)[O-]] |
| t-Bu | KB-1-100 | VAL-1-28 | MPB-1-36 | MPB-1-49 (80 ± 12) | MPB-1-57 (60 ± 7) | KB-1-135 (72 ± 7) | MPB-1-55 (96 ± 7) |
| | (121 ± 3) | (106 ± 20) | (94 ± 12) | | | | |
| Ph | | | | | | | TZ-1-13F3 (44 ± 3) |

Varying the position of the substituent in the aromatic ring (Table 3) also plays significant role in cytotoxicity. Notably, besides the proximity of the substituent to the α-benzylic carbon atom, the other factor that appears to play the role is susceptibility to potential acid-induced cleavage (and cancer cells are known to have slightly acidic pH), which is governed by the stability of a carbocation. This may be the reason why the meta-methoxy derivative (SS-1-11) exhibits the lowest IC₅₀ value of the three derivatives, while the IC50 of the ortho-methoxy derivative (MPB-1-36) is substantially lower than that of the para-methoxy compound (MPB-1-43/KB-1-55), for in the first case the methoxy group is much closer to the benzylic α-carbon.

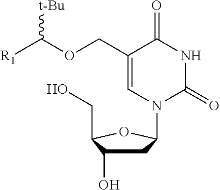

TABLE 2
Effect of the MeO position in T-nucleoside derivatives on
viability of MCF7 cells from the MTT assay: compound (IC₅₀ in μM)

| 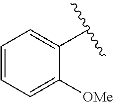 [SMILES: *c1ccccc1OC] | 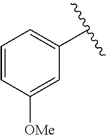 [SMILES: *c1cccc(OC)c1] | 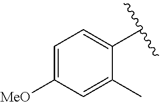 [SMILES: *c1ccc(OC)cc1C] |
|---|---|---|
| MPB-1-36 (94 ± 12) | SS-1-11 (66 ± 10) | KB-1-55 (183 ± 20) |

Cytotoxicity studies of the second generation T-derivatives (Table 4) with diversification at 4-aryl position have revealed that a large, electron withdrawing substituent in that position provides an excellent drug candidate VAL-1-36/38.

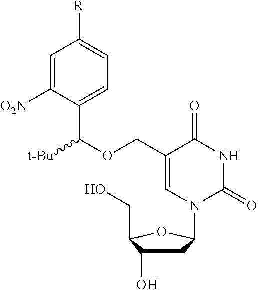

TABLE 4
Cytotoxicity data (MTT in MCF7 breast cancer cells) of the second generation T-nucleoside analogs

| para- substituent | I | 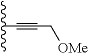 [SMILES: *C#CCOC] | 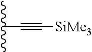 [SMILES: *C#C[Si](C)(C)C] | 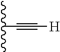 [SMILES: *C#C] | 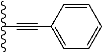 [SMILES: *C#Cc1ccccc1] | 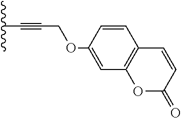 [SMILES: *C#CCOc1ccc2ccc(=O)oc2c1] | 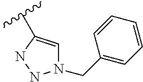 [SMILES: *C1=CN(Cc2ccccc2)NN1] |
|---|---|---|---|---|---|---|---|
| Compound | MPB-1-33 | VAL-1-48 | VAL-1-49 | VAL-1-50 | VAL-1-36/38 | MPB-1-41 | 52 |
| IC₅₀ (μM) | 56 ± 6 | 74 ± 7 | 29 ± 3 | 89 ± 14 | 9 ± 1 | 16 ± 3 | 18 ± 2 |

Nucleobases, on the other hand, have demonstrated slightly different trend, indicating the greatest activity for α-neo-pentyl derivative SL-1-7 (Table 5).

TABLE 5
Cytotoxicity data (MTT in MCF7 breast cancer cells) of T-nucleobase
analogs (IC₅₀ in μM)

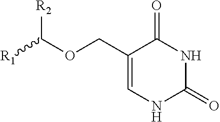

| | R₁ | | |
|---|---|---|---|
| R₂ | i-Pr | t-Bu | neo-Am |
| 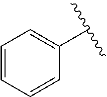 [SMILES: *c1ccccc1] | KB-1-1 (>200) | KB-1-23 (59 ± 2) | SL-1-7 (37 ± 9) |
| 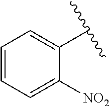 [SMILES: *c1ccccc1[N+](=O)[O-]] | | KB-1-17 (81 ± 10 | |

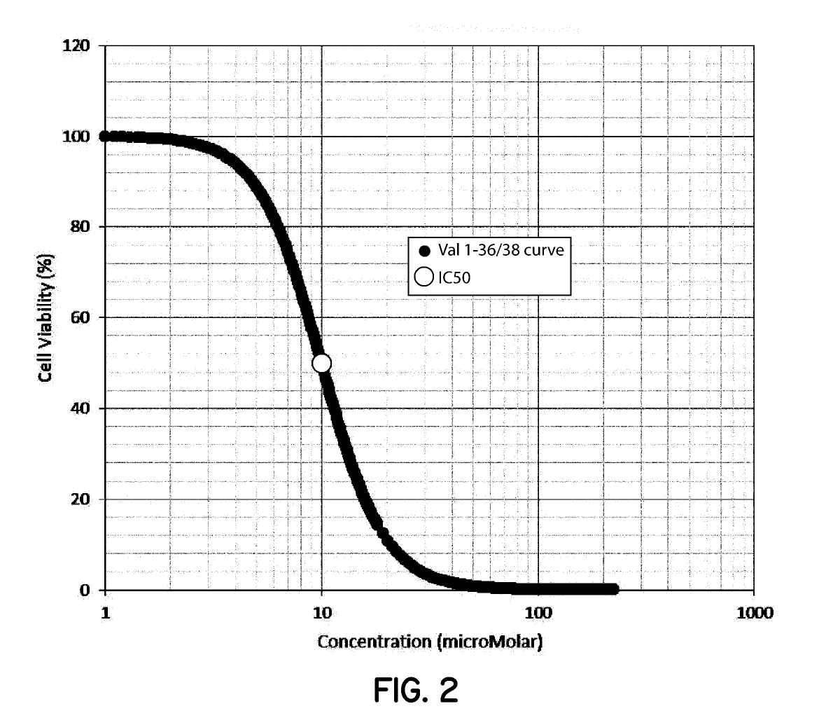

A representative IC₅₀ curve for the second generation lead compound (VAL-1-36/38) is provided in FIG. 2.

An exemplary pharmacophore for MCF7 breast cancer as inferred from SAR studies is depicted below.

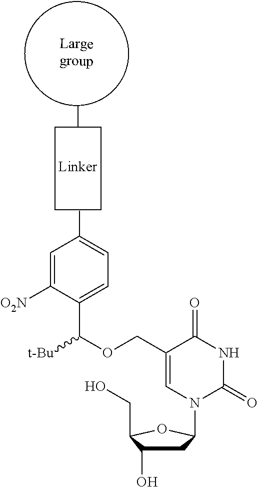

### Example 2

### NCI In Vitro Cancer Screen

Four of the preliminary lead compounds: three nucleosides (JT-1-10, VAL-1-15/42, VAL-1-16) and one nucleobase (KB-1-17)—were submitted to the National Cancer Institute for the in vitro cell line screening project (IVCLSP). The screening consisted in the evaluation of this compound against the 60 cell lines at a single dose of 10 μM.

The human tumor cell lines of the cancer screening panel are grown in RPMI 1640 medium containing 5% fetal bovine serum and 2 mM L-glutamine. For a typical screening experiment, cells are inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO₂, 95% air and 100% relative humidity for 24 h prior to addition of experimental drugs.

After 24 h, two plates of each cell line are fixed in situ with TCA, to represent a measurement of the cell population for each cell line at the time of drug addition (Tz). Experimental drugs are solubilized in dimethyl sulfoxide at 400-fold the desired final maximum test concentration and stored frozen prior to use. At the time of drug addition, an aliquot of frozen concentrate is thawed and diluted to twice the desired final maximum test concentration with complete medium containing 50 μg/ml gentamicin. Additional four, 10-fold or ½ log serial dilutions are made to provide a total of five drug concentrations plus control. Aliquots of 100 μl of these different drug dilutions are added to the appropriate microtiter wells already containing 100 μl of medium, resulting in the required final drug concentrations.

Following drug addition, the plates are incubated for an additional 48 h at 37° C., 5% CO₂, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA). Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Three dose response parameters are calculated for each experimental agent. Growth inhibition of 50% (GI₅₀) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz. The LC₅₀ (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

Growth Inhibition for JT-1-10

It turned out, that the initial lead compound is active against a variety of cancer cell lines, including leukemia, non-small cell lung, central nervous system, renal, and breast.

TABLE 5
NCI-IVCLSP results on GI values for JT-1-10, VAL-1-15, VAL-1-16, and KB-1-17

| JT-1-10 | | VAL-1-15 | | VAL-1-16 | | KB-1-17 | |
|---|---|---|---|---|---|---|---|
| Cancer | Growth | Cancer | Growth | Cancer | Growth | Cancer | Growth |
| Cell Line | Percent | Cell Line | Percent | Cell Line | Percent | Cell Line | Percent |
| Leukemia | 90 | Non-Small | 73 | CNS | 92 | Non-Small | 68 |
| RPMI-8226 | | Cell Lung | | SNB-75 | | Cell Lung | |
| | | HOP-92 | | | | HOP-92 | |
| Non-Small | 89 | CNS | 84 | Melanoma | 90 | CNS | 91 |
| Cell Lung | | SNB-75 | | UACC-62 | | SF-268 | |
| HOP-92 | | | | | | | |
| Non-Small | 91 | Renal | 88 | Renal | 92 | Prostate | 93 |
| Cell Lung | | CAKI-1 | | A498 | | PC-3 | |
| NCI-H522 | | | | | | | |
| CNS | 65 | Prostate | 91 | | | | |
| SNB-75 | | PC-3 | | | | | |
| Renal | 84 | | | | | | |
| TK-10 | | | | | | | |
| Renal | 88 | | | | | | |
| UO-31 | | | | | | | |
| Breast | 87 | | | | | | |
| MSF7 | | | | | | | |

### Example 2

### Protein Extraction and Western Blot Analysis for Drug-Treated MCF7 Cells Detecting Double-Strand Break Marker γ-H2AX

Confluent MCF7 cells were treated with the second generation drug candidate (VAL-1-36/36) at a concentration of ½ the IC₅₀ value (4.5 μM) and were incubated for 16 hours prior to protein extraction. Vehicle controls were simultaneously prepared by treatment with DMSO.

Nuclear proteins from both the drug-treated and control samples were conducted using NE-PER Nuclear and Cytoplasmic Extraction Kit (Thermo Scientific) using the manufacturer's protocol. In detail, cells were harvested by trypsin-EDTA and centrifuged at 500×g for 5 minutes. The cells were washed to remove traces of trypsin by suspending in 1×PBS, followed by centrifugation at 500×g for 2-3 minutes. The supernatant was removed leaving the cell pellet as dry as possible. A 200 μL volume of ice-cold cytoplasmic extraction reagent I (CER-I), treated Halt™ Protease & Phosphatase Inhibitor Cocktail (Thermo Scientific), was added to the pellet. The cell pellet was suspended by vortexing vigorously for 15 seconds and then incubated on ice for 10 minutes. The mixture is then treated with 11 μL of ice-cold cytoplasmic extraction reagent II (CER-II) and mixed by vortexing on the highest setting for 5 seconds followed by incubation on ice of one minute to allow complete release of cytoplasmic contents. The mixture is then vortexed for 5 seconds followed by centrifugation for 5 minutes at maximum speed (16,000×g) in a microcentrifuge. The supernatant, containing the cytoplasmic extract is immediately transferred to a pre-chilled tube and placed on ice until storage. The insoluble pellet, containing the nuclei is suspended in ice-cold nuclear extraction reagent (NER), similarly treated with Halt™ Protease & Phosphatase Inhibitor Cocktail (Thermo Scientific). The mixture is vortexed for 15 seconds at the highest setting and placed on ice for 10 minutes, with the process repeated every 10 minutes for a total of 40 minutes. It is then centrifuged at maximum speed in a microcentrifuge for 10 minutes. The supernatant, containing the nuclear extract is immediately transferred to a pre-chilled microcentrifuge tube and placed on ice until storage at −80° C. All centrifugation steps were performed at 4° C. and all cell samples and extracts were kept on ice.

Protein concentration in each sample and control was determined by Bradford Assay using Coomassie Plus™ (Bradford) Assay Kit (Thermo Scientific), applying the manufacturer's instruction. In detail, 1,000-25 μg/mL concentrations of Albumin Standard by diluting a 2.0 mg/mL stock solution in deionized water, accordingly. The samples were diluted 2× prior to the assay. A volume of 10 μL standard or unknown sample were pipetted into the appropriate wells in a 96-well plate. A volume of 250 μL of the Coomassie Plus Reagent was added to each well and mixed by shaking in a plate shaker for 30 seconds followed by incubation for 10 minutes at RT. The absorbance is then measured at 595 nm using a Synergy 4 plate reader (Biotek). The 595 nm measurement for the blank (0 μg/mL protein) was subtracted from the measurements of all other individual standards and unknown sample measurements. A standard curve is prepared by plotting the Blank-corrected measurement for each BSA standard vs its concentration in μg/mL. The standard curve is used to determine the protein concentration of each unknown sample.

For Western Blot Analysis, 20 μg protein from the nuclear extract of each sample and control is loaded in a 10% ExpressPlus™ SDS-PAGE mini-gel (GenScript) and electrophoresed for 1 hour to separate the component proteins. The proteins are then transferred into a PVDF membrane using the iBlot Dry Blotting system, conducted at 20 V and 7 minute run time. Following transfer, the gel is treated with Coomassie Blue to ensure complete transfer of proteins. The PVDF membrane was wetted in PBS for several minutes. The blocking step was conducted by immersing the membrane in 10 mL Odyssey Blocking Buffer (Licor), with continuous shaking for 1 hour. A 1:1000 dilution of the primary antibodies, Rabbit Anti-Histone H2A.X Ab (Cell Signalling Technology) and Mouse Anti 8-Actin Ab (GenScript), are prepared in 7 mL Odyssey Blocking Buffer. 8-Actin, which is present in cells in high levels is used as the loading control, the signal of which is used to normalize the signal of the protein of interest. The membrane is incubated in the diluted primary antibody solution for one hour, with shaking. Following incubation, the membrane is washed 4× for 5 minutes each at RT in 15 mL PBS+0.1% Tween 20 (Fisher Scientific) with gentle shaking. The fluorescently labeled secondary antibodies, IRDye 800CW Goat anti-Rabbit antibody (1:15000 dilution) and IRDye 680RD Goat anti-Mouse Antibody (1:20000 dilution) are prepared in 10 mL Odyssey Blocking buffer with 0.1% Tween 20 and 0.01% SDS (Fisher Scientific), ensuring minimal exposure to light. After washing with PBS, the membrane was incubated in the secondary antibody solution for 30 minutes at RT with gentle shaking. The membrane is then washed 4× for 5 minutes each with 15 mL PBS+0.1% Tween 20, with gentle shaking and protected from light. To remove residual Tween 20, the membrane is washed with PBS prior to imaging. The membrane is scanned using the Odyssey Infrared Imager (Licor) using the 700 nm channel to detect for 8-Actin and the 800 nm channel to detect for γ-H2AX. The intensity of each band was measured using the ImageQuant 5.0 software. All experiments are conducted at n=2.

Result

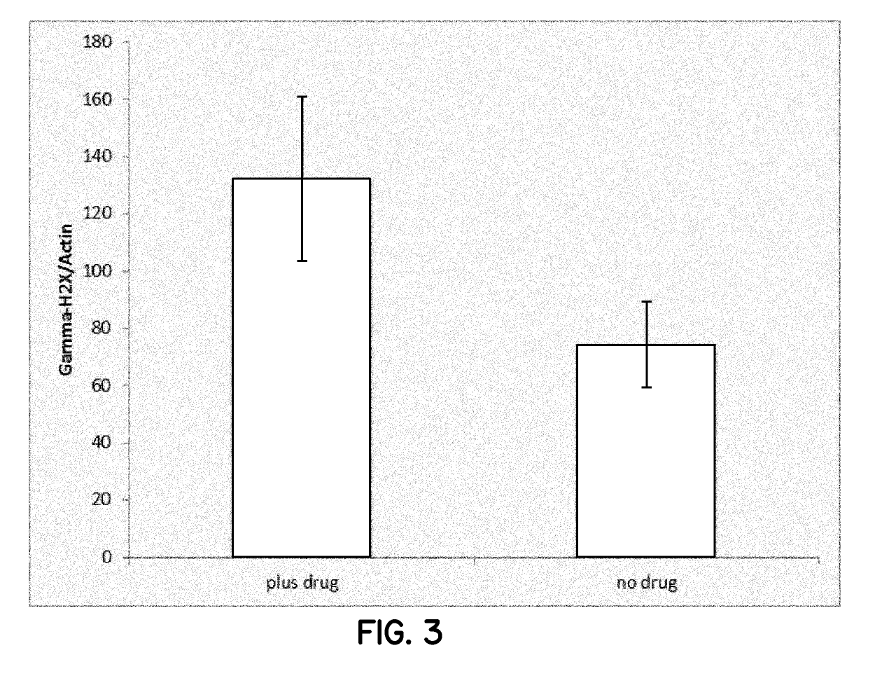

The data shown in the FIG. 3 was obtained from two trials and demonstrate that VL-1-36/38 causes a DNA damage response. This result, as well as the MTT Cell Viability and the SRB Cell Growth Inhibition assays described above support the following hypothesis of the mechanism of action of the drug candidates, according to which they are metabolized into a 5′-triphosphate followed by its incorporation into DNA replication fork and halting further addition of natural nucleotides, so the cell division will not pass the restriction checkpoint, which will prevent the entire process from taking place (Scheme 16).

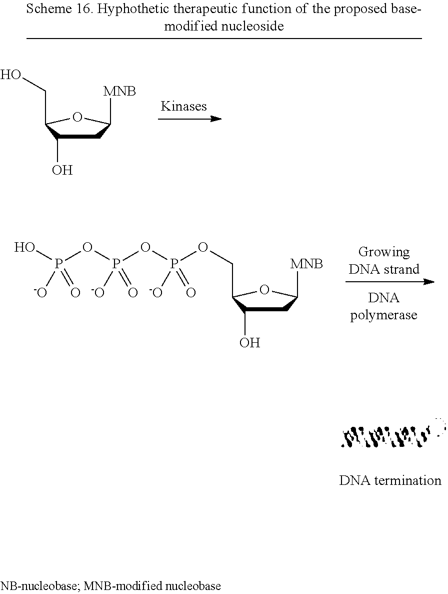

Although the alternative pathways cannot be ruled out, based on the data we have so far, it does appear that the mechanism in Scheme 16 is the most plausible, since it explains the fact that only those modifications on the nucleobase that result in complete DNA termination upon a single incorporation event^{14,15} show significant biological activity, causing cell death (as indicated by the MTT assay), cell growth inhibition (as indicated by SRB assay), and signaling from double-strand break marker γ-H2AX.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative example shown and described. Accordingly, departures may be made from such details without departing from the scope or spirit of the general inventive concept.

1. (canceled)

2. A cancer chemotherapeutic composition comprising:
at least one chemotherapeutic agent derived from a nucleoside or a nucleobase; and
a drug delivery system, wherein the chemotherapeutic agent is selected from the group consisting of: 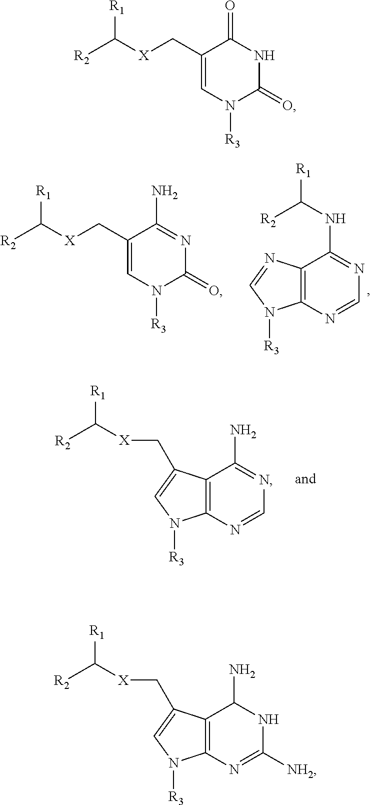
wherein x is one of O, NH, NR, or S;
R₁ is one of a methyl (“Me”), an ethyl (“Et”), an isopropyl (“i-Pr”), tert-butyl (“t-Bu”), neo-pentyl (“neo-Am”), n-hexane (“n-Hex”), cyclohexane (“Cy”), or 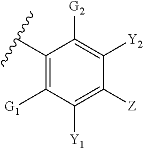
wherein G_{1,2}, Y_{1,2}, and Z are each one of H, Cl, Br, I, methoxy (OMe), or an electron withdrawing group (EWG) that is selected from the group consisting of NO₂, CN, CF₃, COMe, SO₂Me, CCR;
R₂ is one of i-Pr, t-Bu, neo-Am, n-Hex, Cy, or 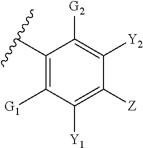
wherein G_{1,2}, Y_{1,2}, and Z are each one of H, Cl, Br, I, OMe, or an EWG that is selected from the group consisting of NO₂, CN, CF₃, COMe, SO₂Me, CCR; and
R₃ is either H or

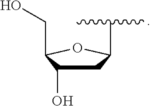

3. The composition of claim 1 wherein the chemotherapeutic agent is selected from the group consisting essentially of KB-1-17, KB-1-1, KB-1-23, SL-1-7, VAL-1-10, MPB-1-34, JT-1-10, VAL-1-15, KB-1-10, MPB-1-35, VAL-1-16, KB-1-55, VAL-1-9, MPB-1-19, JT-1-14, MPB-1-12, MPB-1-27, VAL-1-13, and JT-1-18.

4. The composition of claim 2 wherein the drug delivery system includes β-cyclodextrin.

5. The composition of claim 2 wherein the at least one chemotherapeutic agent is conjugated to the drug delivery system.

6. The composition of claim 5 wherein the chemotherapeutic agent is conjugated to the drug delivery system by an acid labile linker.

7. The composition of claim 6 wherein the acid labile linker is one an acetyl linker or a hydrazone linker.

8. The composition of claim 2 further comprising a targeting ligand.

9. The composition of claim 6 wherein the targeting ligand is conjugated to at least one of the chemotherapeutic agent or the drug delivery system.

10. The composition of claim 8 wherein the targeting ligand is selected from the group consisting of folate, transferrin, RGD peptide, anisimide, and a cancer specific antibody or antibody fragment.

11. The composition of claim 2 wherein the composition is in the form of a nanoparticle having a diameter of at least about 8 nm.

12. The composition of claim 10 wherein the nanoparticle has a diameter of less than about 100 nm.

13. A nucleobase having is selected from the group consisting of: 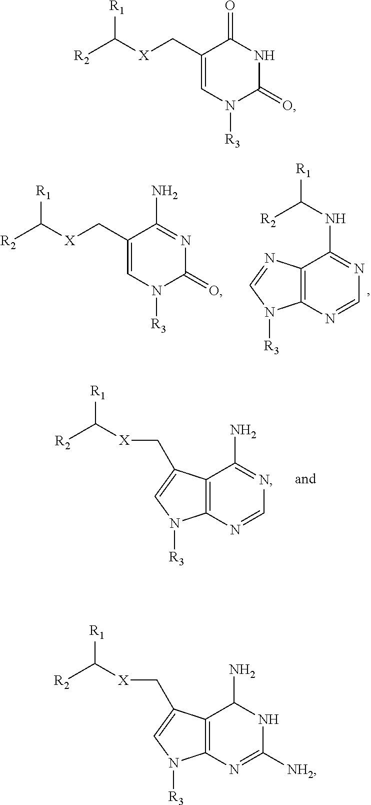
wherein x is one of O, NH, NR, or S;
R₁ is one of a methyl (“Me”), an ethyl (“Et”), an isopropyl (“i-Pr”), tert-butyl (“t-Bu”), neo-pentyl (“neo-Am”), n-hexane (“n-Hex”), cyclohexane (“Cy”), or 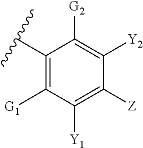
wherein G_{1,2}, Y_{1,2}, and Z are each one of H, Cl, Br, I, methoxy (OMe), or an electron withdrawing group (EWG) that is selected from the group consisting of NO₂, CN, CF₃, COMe, SO₂Me, CCR;
R₂ is one of i-Pr, t-Bu, neo-Am, n-Hex, Cy, or 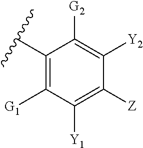
wherein G_{1,2}, Y_{1,2}, and Z are each one of H, Cl, Br, I, OMe, or an EWG that is selected from the group consisting of NO₂, CN, CF₃, COMe, SO₂Me, CCR; and
R₃ is either H or

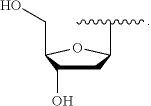

14. A method of treating cancer in a subject comprising an amount of the composition from claim 2 in an amount effective to treat the cancer.
